SEQUENCE LISTING (109 sequences)

SEQ ID NO: 1 - protein, 112 aa, Homo sapiens
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 2 - protein, 106 aa, synthetic construct
  REGION 1..106: note = Core Domain (R21X, N56X, S64X, D103X)
CPLGPGRCCRLHTVXASLEDLGWADWVLSPREVQVTMCIGACPSQFRAAXMHAQIKTXLHRLKPDTVPAPCCVPASYNPMVLIQKTDTGVSLQTYDXLLAKDCHCI

SEQ ID NO: 3 - protein, 5 aa, synthetic construct
  REGION 1..5: note = N-terminal extension domain I
WSNST

SEQ ID NO: 4 - skipped

SEQ ID NO: 5 - protein, 4 aa, synthetic construct
  REGION 1..4: note = N-terminal extension domain III
NGDH

SEQ ID NO: 6 - protein, 106 aa, synthetic construct
  REGION 1..106: note = Core Domain(R21N)
CPLGPGRCCRLHTVNASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 7 - protein, 106 aa, synthetic construct
  REGION 1..106: note = Core Domain(N56L)
CPLGPGRCCRLHTVRASLEDLGWADWVLSPREVQVTMCIGACPSQFRAALMHAQIKTSLHRLKPDTVPAPCCVPASYNPMVLIQKTDTGVSLQTYDDLLAKDCHCI

SEQ ID NO: 8 - protein, 106 aa, synthetic construct
  REGION 1..106: note = Core Domain(S64K)
CPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 9 - protein, 106 aa, synthetic construct
  REGION 1..106: note = Core Domain(S64R)
CPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 10 - protein, 106 aa, synthetic construct
  REGION 1..106: note = Core Domain(S64N)
CPLGPGRCCRLHTVRASLEDLGWADWVLSPREVQVTMCIGACPSQFRAANMHAQIKTNLHRLKPDTVPAPCCVPASYNPMVLIQKTDTGVSLQTYDDLLAKDCHCI

SEQ ID NO: 11 - protein, 106 aa, synthetic construct
  REGION 1..106: note = Core Domain(S64D)
CPLGPGRCCRLHTVRASLEDLGWADWVLSPREVQVTMCIGACPSQFRAANMHAQIKTDLHRLKPDTVPAPCCVPASYNPMVLIQKTDTGVSLQTYDDLLAKDCHCI

SEQ ID NO: 12 - protein, 106 aa, synthetic construct
  REGION 1..106: note = Core Domain(S64E)
CPLGPGRCCRLHTVRASLEDLGWADWVLSPREVQVTMCIGACPSQFRAANMHAQIKTELHRLKPDTVPAPCCVPASYNPMVLIQKTDTGVSLQTYDDLLAKDCHCI

SEQ ID NO: 13 - protein, 106 aa, synthetic construct
  REGION 1..106: note = Core Domain(S64C)
CPLGPGRCCRLHTVRASLEDLGWADWVLSPREVQVTMCIGACPSQFRAANMHAQIKTCLHRLKPDTVPAPCCVPASYNPMVLIQKTDTGVSLQTYDDLLAKDCHCI

SEQ ID NO: 14 - protein, 106 aa, synthetic construct
  REGION 1..106: note = Core Domain(S64L)
CPLGPGRCCRLHTVRASLEDLGWADWVLSPREVQVTMCIGACPSQFRAANMHAQIKTLLHRLKPDTVPAPCCVPASYNPMVLIQKTDTGVSLQTYDDLLAKDCHCI

SEQ ID NO: 15 - protein, 106 aa, synthetic construct
  REGION 1..106: note = Core Domain(D103L)
CPLGPGRCCRLHTVRASLEDLGWADWVLSPREVQVTMCIGACPSQFRAANMHAQIKTSLHRLKPDTVPAPCCVPASYNPMVLIQKTDTGVSLQTYDLLLAKDCHCI

SEQ ID NO: 16 - protein, 106 aa, synthetic construct
  REGION 1..106: note = Core Domain(N56C, D103C)
CPLGPGRCCRLHTVRASLEDLGWADWVLSPREVQVTMCIGACPSQFRAACMHAQIKTSLHRLKPDTVPAPCCVPASYNPMVLIQKTDTGVSLQTYDCLLAKDCHCI

SEQ ID NO: 17 - protein, 106 aa, synthetic construct
  REGION 1..106: note = Core Domain(N56S, D103S)
CPLGPGRCCRLHTVRASLEDLGWADWVLSPREVQVTMCIGACPSQFRAASMHAQIKTSLHRLKPDTVPAPCCVPASYNPMVLIQKTDTGVSLQTYDSLLAKDCHCI

SEQ ID NO: 18 - protein, 106 aa, synthetic construct
  REGION 1..106: note = Core Domain(R21N, S64R)
CPLGPGRCCRLHTVNASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 19 - protein, 106 aa, synthetic construct
  REGION 1..106: note = Core Domain(R21N, S64K)
CPLGPGRCCRLHTVNASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 20 - protein, 106 aa, synthetic construct
  REGION 1..106: note = Core Domain(human GDF15 7-112aa)
CPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 21 - protein, 111 aa, synthetic construct
  REGION 1..111: note = GDF15 variant (delta N3, WS insertion, G4N, D5S, H6T/ FM1)
WSNSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 22 - protein, 109 aa, synthetic construct
  REGION 1..109: note = GDF15 variant (delta N3, G4N, D5S, H6T / FM2)
NSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 23 - protein, 110 aa, synthetic construct
  REGION 1..110: note = GDF15 variant (delta N2, R21N / FM3)
NGDHCPLGPGRCCRLHTVNASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 24 - protein, 110 aa, synthetic construct
  REGION 1..110: note = GDF15 variant (delta N2, N56L / FM4-3)
NGDHCPLGPGRCCRLHTVRASLEDLGWADWVLSPREVQVTMCIGACPSQFRAALMHAQIKTSLHRLKPDTVPAPCCVPASYNPMVLIQKTDTGVSLQTYDDLLAKDCHCI

SEQ ID NO: 25 - protein, 110 aa, synthetic construct
  REGION 1..110: note = GDF15 variant (delta N2, S64K / FM5)
NGDHCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 26 - protein, 111 aa, synthetic construct
  REGION 1..111: note = GDF15 variant (delta N3, WS insertion, G4N, D5S, H6T, S64K / FM6)
WSNSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 27 - protein, 109 aa, synthetic construct
  REGION 1..109: note = GDF15 variant (delta N3, G4N, D5S, H6T, S64K / FM7)
NSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 28 - protein, 110 aa, synthetic construct
  REGION 1..110: note = GDF15 variant (delta N2, S64R / FM9)
NGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 29 - protein, 111 aa, synthetic construct
  REGION 1..111: note = GDF15 variant (delta N3, WS insertion, G4N, D5S, H6T, S64R/ FM10)
WSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 30 - protein, 109 aa, synthetic construct
  REGION 1..109: note = GDF15 variant (delta N3, G4N, D5S, H6T, S64R/ FM11)
NSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 31 - protein, 110 aa, synthetic construct
  REGION 1..110: note = GDF15 variant (delta N2, S64N/ FM13)
NGDHCPLGPGRCCRLHTVRASLEDLGWADWVLSPREVQVTMCIGACPSQFRAANMHAQIKTNLHRLKPDTVPAPCCVPASYNPMVLIQKTDTGVSLQTYDDLLAKDCHCI

SEQ ID NO: 32 - protein, 110 aa, synthetic construct
  REGION 1..110: note = GDF15 variant (delta N2,S64D/ FM14)
NGDHCPLGPGRCCRLHTVRASLEDLGWADWVLSPREVQVTMCIGACPSQFRAANMHAQIKTDLHRLKPDTVPAPCCVPASYNPMVLIQKTDTGVSLQTYDDLLAKDCHCI

SEQ ID NO: 33 - protein, 110 aa, synthetic construct
  REGION 1..110: note = GDF15 variant (delta N2,S64E/ FM15)
NGDHCPLGPGRCCRLHTVRASLEDLGWADWVLSPREVQVTMCIGACPSQFRAANMHAQIKTELHRLKPDTVPAPCCVPASYNPMVLIQKTDTGVSLQTYDDLLAKDCHCI

SEQ ID NO: 34 - protein, 110 aa, synthetic construct
  REGION 1..110: note = GDF15 variant (delta N2,S64L / FM16)
NGDHCPLGPGRCCRLHTVRASLEDLGWADWVLSPREVQVTMCIGACPSQFRAANMHAQIKTLLHRLKPDTVPAPCCVPASYNPMVLIQKTDTGVSLQTYDDLLAKDCHCI

SEQ ID NO: 35 - protein, 110 aa, synthetic construct
  REGION 1..110: note = GDF15 variant (delta N2, D103L / FM4-2)
NGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDLLLAKDCHCI

SEQ ID NO: 36 - protein, 110 aa, synthetic construct
  REGION 1..110: note = GDF15 variant (delta N2, N56C, D103C / FM4)
NGDHCPLGPGRCCRLHTVRASLEDLGWADWVLSPREVQVTMCIGACPSQFRAACMHAQIKTSLHRLKPDTVPAPCCVPASYNPMVLIQKTDTGVSLQTYDCLLAKDCHCI

SEQ ID NO: 37 - protein, 110 aa, synthetic construct
  REGION 1..110: note = GDF15 variant (delta N2, N56S, D103S/ FM4-1)
NGDHCPLGPGRCCRLHTVRASLEDLGWADWVLSPREVQVTMCIGACPSQFRAASMHAQIKTSLHRLKPDTVPAPCCVPASYNPMVLIQKTDTGVSLQTYDSLLAKDCHCI

SEQ ID NO: 38 - protein, 110 aa, synthetic construct
  REGION 1..110: note = GDF15 variant (delta N2, R21N, S64K / FM8)
NGDHCPLGPGRCCRLHTVNASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 39 - protein, 110 aa, synthetic construct
  REGION 1..110: note = GDF15 variant (delta N2, R21N, S64R / FM12)
NGDHCPLGPGRCCRLHTVNASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 40 - protein, 110 aa, synthetic construct
  REGION 1..110: note = GDF15 (delta N2)
NGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 41 - protein, 227 aa, synthetic construct
  REGION 1..227: note = Human IgG1 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 217 aa, synthetic construct
  REGION 1..217: note = Hinge deleted human IgG1-Fc(E233A, L235A)(T366W)-(A-1)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 217 aa, synthetic construct
  REGION 1..217: note = Hinge deleted human IgG1-Fc(E233A, L235A)(T366S, L368A, Y407V)-(A-2)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 217 aa, synthetic construct
  REGION 1..217: note = Hinge deleted human IgG1-Fc(E233A, L235A, N297A)(T366W)-(B-1)
APALAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 217 aa, synthetic construct
  REGION 1..217: note = Hinge deleted human IgG1-Fc(E233A, L235A, N297A)(T366S, L368A, Y407V)-(B-2)
APALAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 217 aa, synthetic construct
  REGION 1..217: note = Hinge deleted human IgG1-Fc(L234A, L235A, N297A)(T366W)-(C-1)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 217 aa, synthetic construct
  REGION 1..217: note = Hinge deleted human IgG1-Fc(L234A, L235A, N297A)(T366S, L368A, Y407V)-(C-2)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 25 aa, synthetic construct
  REGION 1..25: note = linker-(G4S)5
GGGGSGGGGSGGGGSGGGGSGGGGS

SEQ ID NO: 49 - protein, 352 aa, synthetic construct
  REGION 1..352: note = Hinge deleted human IgG1-Fc(E233A, L235A)(T366W)-(G4S)5-GDF15(delta N2) - FWT
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 50 - protein, 353 aa, synthetic construct
  REGION 1..353: note = Hinge deleted human IgG1-Fc(E233A, L235A)(T366W)-(G4S)5-GDF15 variant (delta N3, WS insertion, G4N, D5S, H6T)-FM1
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSWSNSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 51 - protein, 351 aa, synthetic construct
  REGION 1..351: note = Hinge deleted human IgG1-Fc(E233A, L235A)(T366W)-(G4S)5-GDF15 variant (delta N3, G4N, D5S, H6T)-FM2
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNSTCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 52 - protein, 352 aa, synthetic construct
  REGION 1..352: note = Hinge deleted human IgG1-Fc(E233A, L235A)(T366W)-(G4S)5-GDF15 variant (delta N2, R21N)-FM3
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVNASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 53 - protein, 352 aa, synthetic construct
  REGION 1..352: note = Hinge deleted human IgG1-Fc(E233A, L235A)(T366W)-(G4S)5-GDF15 variant (delta N2, N56L)-FM4-3
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAALMHAQIKTSLHRLKPDTVPAPCCVPASYNPMVLIQKTDTGVSLQTYDDLLAKDCHCI

SEQ ID NO: 54 - protein, 352 aa, synthetic construct
  REGION 1..352: note = Hinge deleted human IgG1-Fc(E233A, L235A)(T366W)-(G4S)5-GDF15 variant (delta N2, S64K)-FM5
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 55 - protein, 353 aa, synthetic construct
  REGION 1..353: note = Hinge deleted human IgG1-Fc(E233A, L235A)(T366W)-(G4S)5-GDF15 variant (delta N3, WS insertion, G4N, D5S, H6T, S64K)-FM6
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSWSNSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 56 - protein, 351 aa, synthetic construct
  REGION 1..351: note = Hinge deleted human IgG1-Fc(E233A, L235A)(T366W)-(G4S)5-GDF15 variant (delta N3, G4N, D5S, H6T, S64K)-FM7
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNSTCPLGPGRCCRLHTVRASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 57 - protein, 352 aa, synthetic construct
  REGION 1..352: note = Hinge deleted human IgG1-Fc(E233A, L235A)(T366W)-(G4S)5-GDF15 variant (delta N2, S64R)-FM9
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 58 - protein, 353 aa, synthetic construct
  REGION 1..353: note = Hinge deleted human IgG1-Fc(E233A, L235A)(T366W)-(G4S)5-GDF15 variant (delta N3, WS insertion, G4N, D5S, H6T, S64R)-FM10
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSWSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 59 - protein, 351 aa, synthetic construct
  REGION 1..351: note = Hinge deleted human IgG1-Fc(E233A, L235A)(T366W)-(G4S)5-GDF15 variant (delta N3, G4N, D5S, H6T, S64R)-FM11
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 60 - protein, 352 aa, synthetic construct
  REGION 1..352: note = Hinge deleted human IgG1-Fc(E233A, L235A)(T366W)-(G4S)5-GDF15 variant (delta N2, S64N/ FM13)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTNLHRLKPDTVPAPCCVPASYNPMVLIQKTDTGVSLQTYDDLLAKDCHCI

SEQ ID NO: 61 - protein, 352 aa, synthetic construct
  REGION 1..352: note = Hinge deleted human IgG1-Fc(E233A, L235A)(T366W)-(G4S)5-GDF15 variant (delta N2,S64D)-FM14
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTDLHRLKPDTVPAPCCVPASYNPMVLIQKTDTGVSLQTYDDLLAKDCHCI

SEQ ID NO: 62 - protein, 352 aa, synthetic construct
  REGION 1..352: note = Hinge deleted human IgG1-Fc(E233A, L235A)(T366W)-(G4S)5-GDF15 variant (delta N2,S64E)-FM15
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTELHRLKPDTVPAPCCVPASYNPMVLIQKTDTGVSLQTYDDLLAKDCHCI

SEQ ID NO: 63 - protein, 352 aa, synthetic construct
  REGION 1..352: note = Hinge deleted human IgG1-Fc(E233A, L235A)(T366W)-(G4S)5-GDF15 variant (delta N2,S64L)-FM16
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTLLHRLKPDTVPAPCCVPASYNPMVLIQKTDTGVSLQTYDDLLAKDCHCI

SEQ ID NO: 64 - protein, 352 aa, synthetic construct
  REGION 1..352: note = Hinge deleted human IgG1-Fc(E233A, L235A)(T366W)-(G4S)5-GDF15 variant (delta N2, D103L)-FM4-2
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDLLLAKDCHCI

SEQ ID NO: 65 - protein, 352 aa, synthetic construct
  REGION 1..352: note = Hinge deleted human IgG1-Fc(E233A, L235A)(T366W)-(G4S)5-GDF15 variant (delta N2, N56C, D103C)-FM4
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAACMHAQIKTSLHRLKPDTVPAPCCVPASYNPMVLIQKTDTGVSLQTYDCLLAKDCHCI

SEQ ID NO: 66 - protein, 352 aa, synthetic construct
  REGION 1..352: note = Hinge deleted human IgG1-Fc(E233A, L235A)(T366W)-(G4S)5-GDF15 variant (delta N2, N56S, D103S)-FM4-1
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAASMHAQIKTSLHRLKPDTVPAPCCVPASYNPMVLIQKTDTGVSLQTYDSLLAKDCHCI

SEQ ID NO: 67 - protein, 352 aa, synthetic construct
  REGION 1..352: note = Hinge deleted human IgG1-Fc(E233A, L235A)(T366W)-(G4S)5-GDF15 variant (delta N2, R21N, S64K)-FM8
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVNASLEDLGWADWVLSPREVQVTMCIGACPSQFRAANMHAQIKTKLHRLKPDTVPAPCCVPASYNPMVLIQKTDTGVSLQTYDDLLAKDCHCI

SEQ ID NO: 68 - protein, 352 aa, synthetic construct
  REGION 1..352: note = Hinge deleted human IgG1-Fc(E233A, L235A)(T366W)-(G4S)5-GDF15 variant (delta N2, R21N, S64R)-FM12
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVNASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 69 - protein, 352 aa, synthetic construct
  REGION 1..352: note = Hinge deleted human IgG1-Fc(E233A, L235A)(T366W)-(G4S)5-GDF15 variant (delta N2, W32F)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADFVLSPREVQVTMCIGACPSQFRAANMHAQIKTSLHRLKPDTVPAPCCVPASYNPMVLIQKTDTGVSLQTYDDLLAKDCHCI

SEQ ID NO: 70 - protein, 352 aa, synthetic construct
  REGION 1..352: note = Hinge deleted human IgG1-Fc(E233A, L235A)(T366W)-(G4S)5-GDF15 variant (delta N2, W32H)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADHVLSPREVQVTMCIGACPSQFRAANMHAQIKTSLHRLKPDTVPAPCCVPASYNPMVLIQKTDTGVSLQTYDDLLAKDCHCI

SEQ ID NO: 71 - protein, 352 aa, synthetic construct
  REGION 1..352: note = Hinge deleted human IgG1-Fc(E233A, L235A)(T366W)-(G4S)5-GDF15 variant (delta N2, W32Y)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADYVLSPREVQVTMCIGACPSQFRAANMHAQIKTSLHRLKPDTVPAPCCVPASYNPMVLIQKTDTGVSLQTYDDLLAKDCHCI

SEQ ID NO: 72 - protein, 352 aa, synthetic construct
  REGION 1..352: note = Hinge deleted human IgG1-Fc(E233A, L235A)(T366W)-(G4S)5-GDF15 variant (delta N2, Q51H)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSHFRAANMHAQIKTSLHRLKPDTVPAPCCVPASYNPMVLIQKTDTGVSLQTYDDLLAKDCHCI

SEQ ID NO: 73 - protein, 352 aa, synthetic construct
  REGION 1..352: note = Hinge deleted human IgG1-Fc(E233A, L235A)(T366W)-(G4S)5-GDF15 variant (delta N2, Q51L)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSLFRAANMHAQIKTSLHRLKPDTVPAPCCVPASYNPMVLIQKTDTGVSLQTYDDLLAKDCHCI

SEQ ID NO: 74 - protein, 352 aa, synthetic construct
  REGION 1..352: note = Hinge deleted human IgG1-Fc(E233A, L235A)(T366W)-(G4S)5-GDF15 variant (delta N2, Q51E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSEFRAANMHAQIKTSLHRLKPDTVPAPCCVPASYNPMVLIQKTDTGVSLQTYDDLLAKDCHCI

SEQ ID NO: 75 - protein, 352 aa, synthetic construct
  REGION 1..352: note = Hinge deleted human IgG1-Fc(E233A, L235A)(T366W)-(G4S)5-GDF15 variant (delta N2, Q51N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSNFRAANMHAQIKTSLHRLKPDTVPAPCCVPASYNPMVLIQKTDTGVSLQTYDDLLAKDCHCI

SEQ ID NO: 76 - protein, 352 aa, synthetic construct
  REGION 1..352: note = Hinge deleted human IgG1-Fc(E233A, L235A)(T366W)-(G4S)5-GDF15 variant (delta N2, Q90H)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIHKTDTGVSLQTYDDLLAKDCHCI

SEQ ID NO: 77 - protein, 352 aa, synthetic construct
  REGION 1..352: note = Hinge deleted human IgG1-Fc(E233A, L235A)(T366W)-(G4S)5-GDF15 variant (delta N2, Q90E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIEKTDTGVSLQTYDDLLAKDCHCI

SEQ ID NO: 78 - protein, 352 aa, synthetic construct
  REGION 1..352: note = Hinge deleted human IgG1-Fc(E233A, L235A)(T366W)-(G4S)5-GDF15 variant (delta N2, Q90K)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIKKTDTGVSLQTYDDLLAKDCHCI

SEQ ID NO: 79 - protein, 352 aa, synthetic construct
  REGION 1..352: note = Hinge deleted human IgG1-Fc(E233A, L235A)(T366W)-(G4S)5-GDF15 variant (delta N2, D93E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTETGVSLQTYDDLLAKDCHCI

SEQ ID NO: 80 - protein, 352 aa, synthetic construct
  REGION 1..352: note = Hinge deleted human IgG1-Fc(E233A, L235A)(T366W)-(G4S)5-GDF15 variant (delta N2, D93L)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTLTGVSLQTYDDLLAKDCHCI

SEQ ID NO: 81 - protein, 352 aa, synthetic construct
  REGION 1..352: note = Hinge deleted human IgG1-Fc(E233A, L235A)(T366W)-(G4S)5-GDF15 variant (delta N2, D93N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTNTGVSLQTYDDLLAKDCHCI

SEQ ID NO: 82 - protein, 352 aa, synthetic construct
  REGION 1..352: note = Hinge deleted human IgG1-Fc(E233A, L235A)(T366W)-(G4S)5-GDF15 variant (delta N2, D93Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTQTGVSLQTYDDLLAKDCHCI

SEQ ID NO: 83 - protein, 352 aa, synthetic construct
  REGION 1..352: note = Hinge deleted human IgG1-Fc(E233A, L235A)(T366W)-(G4S)5-GDF15 variant (delta N2, Q60L)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHALIKTSLHRLKPDTVPAPCCVPASYNPMVLIQKTDTGVSLQTYDDLLAKDCHCI

SEQ ID NO: 84 - protein, 352 aa, synthetic construct
  REGION 1..352: note = Hinge deleted human IgG1-Fc(E233A, L235A)(T366W)-(G4S)5-GDF15 variant (delta N2,Q60N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHANIKTSLHRLKPDTVPAPCCVPASYNPMVLIQKTDTGVSLQTYDDLLAKDCHCI

SEQ ID NO: 85 - protein, 352 aa, synthetic construct
  REGION 1..352: note = Hinge deleted human IgG1-Fc(E233A, L235A)(T366W)-(G4S)5-GDF15 variant (delta N2, S64Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTQLHRLKPDTVPAPCCVPASYNPMVLIQKTDTGVSLQTYDDLLAKDCHCI

SEQ ID NO: 86 - protein, 352 aa, synthetic construct
  REGION 1..352: note = Hinge deleted human IgG1-Fc(E233A, L235A)(T366W)-(G4S)5-GDF15 variant (delta N2, T92S)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKSDTGVSLQTYDDLLAKDCHCI

SEQ ID NO: 87 - protein, 352 aa, synthetic construct
  REGION 1..352: note = Hinge deleted human IgG1-Fc(E233A, L235A)(T366W)-(G4S)5-GDF15 variant (delta N2, T92E)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKEDTGVSLQTYDDLLAKDCHCI

SEQ ID NO: 88 - protein, 352 aa, synthetic construct
  REGION 1..352: note = Hinge deleted human IgG1-Fc(E233A, L235A)(T366W)-(G4S)5-GDF15 variant (delta N2, S97N)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVNLQTYDDLLAKDCHCI

SEQ ID NO: 89 - protein, 352 aa, synthetic construct
  REGION 1..352: note = Hinge deleted human IgG1-Fc(E233A, L235A)(T366W)-(G4S)5-GDF15 variant (delta N2, S97Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVQLQTYDDLLAKDCHCI

SEQ ID NO: 90 - protein, 352 aa, synthetic construct
  REGION 1..352: note = Hinge deleted human IgG1-Fc(E233A, L235A)(T366W)-(G4S)5-GDF15 variant (delta N2,Y101F)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTFDDLLAKDCHCI

SEQ ID NO: 91 - protein, 352 aa, synthetic construct
  REGION 1..352: note = Hinge deleted human IgG1-Fc(E233A, L235A)(T366W)-(G4S)5-GDF15 variant (delta N2, Y101Q)
APALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTQDDLLAKDCHCI

SEQ ID NO: 92 - protein, 17 aa, synthetic construct
  REGION 1..17: note = linker-GS(G4S)3
GSGGGGSGGGGSGGGGS

SEQ ID NO: 93 - protein, 27 aa, synthetic construct
  REGION 1..27: note = linker-GS(G4S)5
GSGGGGSGGGGSGGGGSGGGGSGGGGS

SEQ ID NO: 94 - protein, 37 aa, synthetic construct
  REGION 1..37: note = linker-GS(G4S)7
GSGGGGSGGGGSGGGGSGGGGSGGGGSGGGGSGGGGS

SEQ ID NO: 95 - protein, 26 aa, synthetic construct
  REGION 1..26: note = linker-GS(EEEA)6
GSEEEAEEEAEEEAEEEAEEEAEEEA

SEQ ID NO: 96 - protein, 26 aa, synthetic construct
  REGION 1..26: note = linker-GSGGSS(PT)10
GSGGSSPTPTPTPTPTPTPTPTPTPT

SEQ ID NO: 97 - protein, 27 aa, synthetic construct
  REGION 1..27: note = linker-GS(EAAAK)5
GSEAAAKEAAAKEAAAKEAAAKEAAAK

SEQ ID NO: 98 - protein, 344 aa, synthetic construct
  REGION 1..344: note = Hinge deleted human IgG1-Fc(L234A, L235A, N297A)(T366W)-GS(G4S)3-GDF15 variant (delta N2, S64R / FM9-1)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 99 - protein, 354 aa, synthetic construct
  REGION 1..354: note = Hinge deleted human IgG1-Fc(L234A, L235A, N297A)(T366W)-GS(G4S)5-GDF15 variant (delta N2, S64R / FM9-2)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 100 - protein, 364 aa, synthetic construct
  REGION 1..364: note = Hinge deleted human IgG1-Fc(L234A, L235A, N297A)(T366W)-GS(G4S)7-GDF15 variant (delta N2, S64R / FM9-3)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSGGGGSNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 101 - protein, 353 aa, synthetic construct
  REGION 1..353: note = Hinge deleted human IgG1-Fc(L234A, L235A, N297A)(T366W)-GS(EEEA)6-GDF15 variant (delta N2, S64R / FM9-4)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEEEAEEEAEEEAEEEAEEEAEEEANGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 102 - protein, 353 aa, synthetic construct
  REGION 1..353: note = Hinge deleted human IgG1-Fc(L234A, L235A, N297A)(T366W)-GSGGSS(PT)10-GDF15 variant (delta N2, S64R / FM9-5)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGSSPTPTPTPTPTPTPTPTPTPTNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 103 - protein, 354 aa, synthetic construct
  REGION 1..354: note = Hinge deleted human IgG1-Fc(L234A, L235A, N297A)(T366W)-GS(EAAAK)5-GDF15 variant (delta N2, S64R / FM9-6)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEAAAKEAAAKEAAAKEAAAKEAAAKNGDHCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 104 - protein, 343 aa, synthetic construct
  REGION 1..343: note = Hinge deleted human IgG1-Fc(L234A, L235A, N297A)(T366W)-GS(G4S)3-GDF15 variant (delta N3, G4N, D5S, H6T, S64R/ FM11-1)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 105 - protein, 353 aa, synthetic construct
  REGION 1..353: note = Hinge deleted human IgG1-Fc(L234A, L235A, N297A)(T366W)-GS(G4S)5-GDF15 variant (delta N3, G4N, D5S, H6T, S64R/ FM11-2)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 106 - protein, 363 aa, synthetic construct
  REGION 1..363: note = Hinge deleted human IgG1-Fc(L234A, L235A, N297A)(T366W)-GS(G4S)7-GDF15 variant (delta N3, G4N, D5S, H6T, S64R/ FM11-3)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSGGGGSNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 107 - protein, 352 aa, synthetic construct
  REGION 1..352: note = Hinge deleted human IgG1-Fc(L234A, L235A, N297A)(T366W)-GS(EEEA)6-GDF15 variant (delta N3, G4N, D5S, H6T, S64R/ FM11-4)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEEEAEEEAEEEAEEEAEEEAEEEANSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 108 - protein, 352 aa, synthetic construct
  REGION 1..352: note = Hinge deleted human IgG1-Fc(L234A, L235A, N297A)(T366W)-GSGGSS(PT)10-GDF15 variant (delta N3, G4N, D5S, H6T, S64R/ FM11-5)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSGGSSPTPTPTPTPTPTPTPTPTPTNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

SEQ ID NO: 109 - protein, 353 aa, synthetic construct
  REGION 1..353: note = Hinge deleted human IgG1-Fc(L234A, L235A, N297A)(T366W)-GS(EAAAK)5-GDF15 variant (delta N3, G4N, D5S, H6T, S64R/ FM11-6)
APEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGSEAAAKEAAAKEAAAKEAAAKEAAAKNSTCPLGPGRCCRLHTVRASLEDLGWADWVLSPREVQVTMCIGACPSQFRAANMHAQIKTRLHRLKPDTVPAPCCVPASYNPMVLIQKTDTGVSLQTYDDLLAKDCHCI

(19) United States
(12) Patent Application Publication
(10) Pub. No.: US 2024/0082354 A1
(43) Pub. Date: Mar. 14, 2024

(54) COMPOSITION FOR PREVENTING OR TREATING NON-ALCOHOLIC FATTY LIVER DISEASE OR NON-ALCOHOLIC STEATOHEPATITIS COMPRISING GROWTH DIFFERENTIATION FACTOR-15 VARIANT

(71) Applicant: YUHAN CORPORATION, Seoul (KR)

(72) Inventors: Seyoung LIM, Gyeonggi-do (KR); Ji Eun YANG, Gyeonggi-do (KR); Do Hoon KIM, Gyeonggi-do (KR); Sukyung KIM, Seoul (KR); Bo Ra SIM, Gyeonggi-do (KR); Yunji LEE, Gyeonggi-do (KR); Nayeon YANG, Gyeonggi-do (KR); Junhwan KIM, Seoul (KR)

(21) Appl. No.: 18/513,613

(22) Filed: Nov. 19, 2023

Related U.S. Application Data
(63) Continuation-in-part of application No. PCT/KR2022/007248, filed on May 20, 2022.

(30) Foreign Application Priority Data
May 21, 2021 (KR) 10-2021-0065564

(51) Int. Cl. A61K 38/18; A61P 1/16

(52) U.S. Cl. CPC A61K 38/1841; A61P 1/16

(57) ABSTRACT

The present invention relates to a pharmaceutical composition for the prevention or treatment of non-alcoholic fatty liver disease (NAFLD) or non-alcoholic steatohepatitis (NASH), comprising a GDF15 (growth differentiation factor-15) variant, a long-acting GDF15 fusion protein, or a long-acting GDF15 fusion protein dimer as an active ingredient.

### CROSS-REFERENCE TO RELATED APPLICATIONS

This is a continuation-in-part under 35 USC § 120 of International Patent Application PCT/KR2022/007248 filed May 20, 2022, which claims priority under 35 USC § 119 of Korean Patent Application 10-2021-0065564 filed May 21, 2021. The disclosures of all such applications are hereby incorporated herein by reference, in their respective entireties, for all purposes.

### SEQUENCE LISTING

This application includes an electronically submitted sequence listing in .xml format. The .xml file contains a sequence listing entitled “705PCTCIP_SequenceListing.xml” created on Nov. 19, 2023 and is 164,648 bytes in size. The sequence listing contained in this .xml file is part of the specification and is hereby incorporated by reference herein in its entirety.

### BACKGROUND OF THE INVENTION

### Field of the Invention

The present invention relates to a pharmaceutical composition for the prevention or treatment of non-alcoholic fatty liver disease (NAFLD) or non-alcoholic steatohepatitis (NASH), comprising a GDF15 (growth differentiation factor-15) variant, a long-acting GDF15 fusion protein, or a long-acting GDF15 fusion protein dimer as an active ingredient.

### Description of the Related Art

GDF15, called MIC-1 (macrophage inhibitory cytokine-1), PBMP (placental bone morphogenetic protein), or NAG-1 (nonsteroidal anti-inflammatory drug-activated gene-1), is a protein that is a member of the TGF-β, superfamily (transforming growth factor-beta superfamily).

Recently, it has been reported that GDF15 induces loss of body weight by inhibiting dietary intake through binding to RET (ret proto-oncogene) and GFRAL (GDNF family receptor alpha-like) specifically expressed in brain tissue (Tsai V. W. et al., PLoS One 2013; 8 (2): e55174; U.S. Pat. No. 8,192,735). Moreover, in several studies, administration of GDF15 to a variety of obese animal models demonstrated an excellent weight loss effect, and additionally, metabolic advantages such as lowered blood glucose levels, reduced lipid levels, improved insulin resistance, and the like were observed.

However, wild-type GDF15 has a short half-life in the body, so there is a problem in that the frequency of administration thereof is high when used medically. Accordingly, development of a long-acting formulation for increasing the half-life of GDF15 in the body is underway.

Meanwhile, all fatty liver diseases characterized by fatty liver similar to alcoholic liver disease but occurring in non-drinkers are collectively referred to as non-alcoholic fatty liver disease (NAFLD). Fatty acid synthesis in the liver is always active in patients with non-alcoholic fatty liver disease, and activation of fatty acid synthesis is an important factor in fatty liver formation due to metabolic syndrome.

Non-alcoholic fatty liver disease is broadly divided into simple fatty liver, which is generally considered to have a good prognosis, and non-alcoholic steatohepatitis (NASH), which is judged to have a poor prognosis due to persistent inflammation or fibrosis of the simple fatty liver, and NASH may be said to be a form of severe NAFLD. According to statistics, it has been reported that about 30% of people with fatty liver show signs of NASH, and 10-29% of NASH patients develop cirrhosis, progressing to liver cancer or death. However, despite the fact that non-alcoholic steatohepatitis is a severe disease that progresses to cirrhosis and liver cancer, no drug clinically demonstrated to greatly ameliorate NASH has been reported to date, so research into the prevention or treatment of non-alcoholic steatohepatitis is urgently required.

The present inventors ascertained, during evaluation of the preventive or therapeutic effect on metabolic syndrome using a GDF15 variant exhibiting improved activity by introducing a mutation at a certain position of GDF15, the likelihood of amelioration of non-alcoholic fatty liver disease (NAFLD) and non-alcoholic steatohepatitis (NASH) in a NASH animal model, thus culminating in the present invention.

### CITATION LIST

(Patent Document 1) US 2019-0000923 (2019.01.03)

(Non-Patent Document 1) Li Ding et al., BBRC 2018; 498:388-394

(Non-Patent Document 2) Zhang Zechusan et al., J. Hepatol 2020; 72:976-989

### SUMMARY OF THE INVENTION

It is an object of the present invention to provide a pharmaceutical composition for preventing or treating non-alcoholic fatty liver disease (NAFLD) or non-alcoholic steatohepatitis (NASH), comprising a GDF15 variant, a long-acting GDF15 fusion protein, or a long-acting GDF15 fusion protein dimer as an active ingredient.

In order to accomplish the above object, the present invention provides a pharmaceutical composition for preventing or treating non-alcoholic fatty liver disease (NAFLD) or non-alcoholic steatohepatitis (NASH), which includes a GDF15 (growth differentiation factor-15) variant represented by Formula (I) below, a long-acting GDF15 fusion protein, or a long-acting GDF15 fusion protein dimer as an active ingredient:

N-terminal extension domain−core domain Formula (I)

in Formula (I),

the N-terminal extension domain is a polypeptide including any one amino acid sequence selected from among SEQ ID NOs: 3 to 5; and

the core domain is a polypeptide including the amino acid sequence of SEQ ID NO: 20, or a polypeptide in which any one selected from the group consisting of the 15^{th} amino acid, 50^{th} amino acid, 58^{th} amino acid, 97^{th} amino acid, and combinations thereof in the amino acid sequence of SEQ ID NO: 20 is substituted with another amino acid,

in which arginine (R), which is the 15^{th} amino acid, may be substituted with alanine (A), aspartic acid (D), asparagine (N), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), or valine (V),

asparagine (N), which is the 50^{th} amino acid, may be substituted with alanine, arginine, aspartic acid, cysteine, glutamic acid, glutamine, glycine, histidine, isoleucine, leucine, lysine, methionine, phenylalanine, proline, serine, threonine, tryptophan, tyrosine, or valine,

serine (S), which is the 58^{th} amino acid, may be substituted with alanine, arginine, aspartic acid, asparagine, cysteine, glutamic acid, glutamine, glycine, histidine, isoleucine, leucine, lysine, methionine, phenylalanine, proline, threonine, tryptophan, tyrosine, or valine, or

aspartic acid (D), which is the 97^{th} amino acid, may be substituted with alanine, arginine, cysteine, glutamic acid, glutamine, glycine, histidine, isoleucine, leucine, lysine, methionine, phenylalanine, proline, serine, threonine, tryptophan, tyrosine, or valine.

### BRIEF DESCRIPTION OF THE DRAWINGS

The above and other objects, features, and other advantages of the present invention will be more clearly understood from the following detailed description taken in conjunction with the accompanying drawings, in which:

FIG. 1 shows the results of measurement of functional activity of long-acting GDF15 fusion proteins (dimer, FM9-1, FM9-2, FM9-3, FM9-4, FM9-5, and FM9-6) depending on the linker type and length;

FIG. 2 shows the results of measurement of functional activity of long-acting GDF15 fusion proteins (dimer, FM11-1, FM11-2, FM11-3, FM11-4, FM11-5 and FM11-6) depending on the linker type and length;

FIG. 3 shows the results of measurement of body weight change (%) in a choline-deficient, L-amino-acid-defined, high-fat-diet-induced non-alcoholic steatohepatitis mouse model (CDAHF-diet-induced NASH mouse model) upon repeated administration of a long-acting GDF15 fusion protein (dimer, FM9-6);

FIG. 4 shows the results of measurement of the relative liver weight reduction effect in a CDAHF-diet-induced NASH mouse model upon repeated administration of a long-acting GDF15 fusion protein (dimer, FM9-6) (###p<0.001 vs. normal control (normal vehicle) (Student's t-test) and ***p<0.001, and ****p<0.0001 vs. CDAHF diet control (CDAHF vehicle) (One-way ANOVA));

FIG. 5 shows the results of measurement of the improvement in liver function blood indicators in a CDAHF-diet-induced NASH mouse model upon repeated administration of a long-acting GDF15 fusion protein (dimer, FM9-6) (###p<0.001 vs. normal control (normal vehicle) (Student's t-test) and *p<0.05, and **p<0.01 vs. CDAHF diet control (CDAHF vehicle) (One-way ANOVA));

FIG. 6 shows the results of improved a NAFLD activity score (NAS) in a CDAHF-diet-induced NASH mouse model upon repeated administration of a long-acting GDF15 fusion protein (dimer, FM9-6) (###p<0.001 vs. normal control (normal vehicle) (Student's t-test) and **p<0.01 vs. CDAHF diet control (CDAHF vehicle) (One-way ANOVA));

FIG. 7 shows the results of measurement of individual steatosis and hypertrophy scores in a CDAHF-diet-induced NASH mouse model upon repeated administration of a long-acting GDF15 fusion protein (dimer, FM9-6);

FIG. 8 shows the results of measurement of liver triglyceride levels in a CDAHF-diet-induced NASH mouse model upon repeated administration of a long-acting GDF15 fusion protein (dimer, FM9-6) (###p<0.001 vs. normal control (normal vehicle) (Student's t-test) and ***p<0.001 vs. CDAHF diet control (CDAHF vehicle) (One-way ANOVA));

FIG. 9 shows H&E-stained liver tissue images in a CDAHF-diet-induced NASH mouse model upon repeated administration of a long-acting GDF15 fusion protein (dimer, FM9-6);

FIG. 10 shows the results of analysis of Picrosirius red (PSR) staining in a CDAHF-diet-induced NASH mouse model upon repeated administration of a long-acting GDF15 fusion protein (dimer, FM9-6) (###p<0.001 vs. normal control (normal vehicle) (Student's t-test) and ***p<0.001, and ****p<0.0001 vs. CDAHF diet control (CDAHF vehicle) (One-way ANOVA));

FIG. 11 shows the results of measurement of body weight change (%) in an ob/ob mouse model upon repeated administration of a long-acting GDF15 fusion protein (dimer, FM9-6);

FIG. 12 shows the results of measurement of the effect of reduction of absolute liver weight(g) and relative liver weight (%) in an ob/ob mouse model upon repeated administration of a long-acting GDF15 fusion protein (dimer, FM9-6) (####p<0.0001 vs. normal control (lean vehicle) (Student's t-test) and ****p<0.0001 vs. ob/ob control (ob/ob vehicle) (One-way ANOVA); and

FIG. 13 shows the results of measurement of the improvement in liver function blood indicators in an ob/ob mouse model upon repeated administration of a long-acting GDF15 fusion protein (dimer, FM9-6) (###p<0.001, and ####p<0.0001 vs. normal control (lean vehicle) (Student's t-test) and *p<0.05, **p<0.01, ***p<0.001, and ****p<0.0001 vs. ob/ob control (ob/ob vehicle) (One-way ANOVA).

### DETAILED DESCRIPTION OF THE INVENTION

An aspect of the present invention pertains to a pharmaceutical composition for preventing or treating non-alcoholic fatty liver disease (NAFLD) or non-alcoholic steatohepatitis (NASH), comprising a GDF15 (growth differentiation factor-15) variant, a long-acting GDF15 fusion protein, or a long-acting GDF15 fusion protein dimer as an active ingredient.

Hereinafter, a detailed description will be given of the present invention.

GDF15 Variant

A GDF15 variant represented by Formula (I) below is provided.

N-terminal extension domain−core domain Formula (I)

In Formula (I),

the N-terminal extension domain is a polypeptide including any one amino acid sequence selected from among SEQ ID NOs: 3 to 5; and

the core domain is a polypeptide including the amino acid sequence of SEQ ID NO: 20, or a polypeptide in which any one selected from the group consisting of the 15^{th} amino acid, 50^{th} amino acid, 58^{th} amino acid, 97^{th} amino acid, and combinations thereof in the amino acid sequence of SEQ ID NO: 20 is substituted with another amino acid,

in which arginine (R), which is the 15^{th} amino acid, may be substituted with alanine (A), aspartic acid (D), asparagine (N), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), or valine (V),

asparagine (N), which is the 50^{th} amino acid, may be substituted with alanine, arginine, aspartic acid, cysteine, glutamic acid, glutamine, glycine, histidine, isoleucine, leucine, lysine, methionine, phenylalanine, proline, serine, threonine, tryptophan, tyrosine, or valine,

serine (S), which is the 58^{th} amino acid, may be substituted with alanine, arginine, aspartic acid, asparagine, cysteine, glutamic acid, glutamine, glycine, histidine, isoleucine, leucine, lysine, methionine, phenylalanine, proline, threonine, tryptophan, tyrosine, or valine, or

aspartic acid (D), which is the 97^{th} amino acid, may be substituted with alanine, arginine, asparagine cysteine, glutamic acid, glutamine, glycine, histidine, isoleucine, leucine, lysine, methionine, phenylalanine, proline, serine, threonine, tryptophan, tyrosine, or valine.

As used herein, the term “core domain” refers to a polypeptide having the amino acid sequence from the 7^{th} amino acid to the 112^{th} amino acid in the amino acid sequence of GDF15 of SEQ ID NO: 1, and is a polypeptide including the amino acid sequence of SEQ ID NO: 20 or a polypeptide in which any one selected from the group consisting of the 15^{th} amino acid, 50^{th} amino acid, 58^{th} amino acid, 97^{th} amino acid, and combinations thereof in the amino acid sequence of SEQ ID NO: 20 is substituted with another amino acid. The first core domain may include the amino acid sequence of SEQ ID NO: 2.

Specifically, the core domain may include any one mutation selected from the group consisting of the following mutations (1) to (6):
(1) arginine (R), which is the 15^{th} amino acid in the amino acid sequence of SEQ ID NO: 20, is substituted with asparagine (N);
(2) asparagine (N), which is the 50^{th} amino acid in the amino acid sequence of SEQ ID NO: 20, is substituted with leucine (L);
(3) serine (S), which is the 58^{th} amino acid in the amino acid sequence of SEQ ID NO: 20, is substituted with lysine (K), arginine (R), asparagine (N), aspartic acid (D), glutamic acid (E), cysteine (C), or leucine (L);
(4) aspartic acid (D), which is the 97^{th} amino acid in the amino acid sequence of SEQ ID NO: 20, is substituted with leucine (L);
(5) asparagine (N), which is the 50^{th} amino acid, and aspartic acid (D), which is the 97^{th} amino acid, in the amino acid sequence of SEQ ID NO: 20, are substituted with cysteine (C) or serine (S); and
(6) arginine (R), which is the 15^{th} amino acid in the amino acid sequence of SEQ ID NO: 20, is substituted with asparagine (N), and serine (S), which is the 58^{th} amino acid, is substituted with lysine (K) or arginine (R).

Here, the core domain may include any one amino acid sequence selected from among SEQ ID NOs: 6 to 19.

The N-terminal extension domain is a domain bound to the N-terminus of the core domain, and may be a polypeptide including any one amino acid sequence selected from among SEQ ID NOs: 3 to 5.

As used herein, the expression “ΔN2” may also be represented as “delta N2”, and means that the first and second amino acids in the amino acid sequence of human GDF15 set forth in SEQ ID NO: 1 are deleted. ΔN2 may be represented as “NGDH” when expressed as an N-terminal extension domain.

As used herein, the expression “ΔN3, WS insertion, G4N, D5S, H6T” may also be represented as “delta N3, WS insertion, G4N, D5S, H6T”, and means that the first to third amino acids in the amino acid sequence of human GDF15 set forth in SEQ ID NO: 1 are deleted, tryptophan and serine are inserted at the deleted positions, glycine, which is the fourth amino acid, is substituted with asparagine, aspartic acid, which is the fifth amino acid, is substituted with serine, and histidine, which is the sixth amino acid, is substituted with threonine, respectively. The ΔN3, WS insertion, G4N, D5S, and H6T may be represented as “WSNST” when expressed as an N-terminal extension domain.

As used herein, the expression “ΔN3, G4N, D5S, H6T” may also be represented as “delta N3, G4N, D5S, H6T”, and means that the first to third amino acids in the amino acid sequence of human GDF15 set forth in SEQ ID NO: 1 are deleted, glycine, which is the fourth amino acid, is substituted with asparagine, aspartic acid, which is the fifth amino acid, is substituted with serine, and histidine, which is the sixth amino acid, is substituted with threonine, respectively. The ΔN3, G4N, D5S, and H6T may be represented as “NST” when expressed as an N-terminal extension domain.

The GDF15 variant may include an N-terminal extension domain including the amino acid sequence set forth in SEQ ID NO: 3 and a core domain including any one amino acid sequence selected from among SEQ ID NOs: 6 to 20. In addition, the GDF15 variant may include an N-terminal extension domain including the amino acid sequence set forth in SEQ ID NO: 4 and a core domain including any one amino acid sequence selected from among SEQ ID NOs: 6 to 20. Moreover, the GDF15 variant may include an N-terminal extension domain including the amino acid sequence set forth in SEQ ID NO: 5 and a core domain including any one amino acid sequence selected from among SEQ ID NOs: 6 to 19.

Preferably, the GDF15 variant includes an N-terminal extension domain including the amino acid sequence set forth in SEQ ID NO: 3 and a core domain including the amino acid sequence set forth in SEQ ID NO: 8, 9, or 20. In addition, the GDF15 variant includes an N-terminal extension domain including the amino acid sequence set forth in SEQ ID NO: 4 and a core domain including the amino acid sequence set forth in SEQ ID NO: 8, 9, or 20. In addition, the GDF15 variant includes an N-terminal extension domain including the amino acid sequence set forth in SEQ ID NO: 5 and a core domain including any one amino acid sequence selected from among SEQ ID NOs: 6, 7, and 10 to 19. Here, the GDF15 variant may include any one amino acid sequence selected from among SEQ ID NOs: 21 to 39.

Long-Acting GDF15 Fusion Protein

A long-acting GDF15 fusion protein is configured such that the GDF15 variant and a human IgG Fc or a variant thereof are bound to each other.

The human IgG Fc or the variant thereof may be Fc of IgG1, IgG2, IgG3, or IgG4, or a variant thereof. Specifically, the human IgG1 Fc or the variant thereof may be a human IgG1 Fc or a variant thereof, and the human IgG1 Fc may include the amino acid sequence set forth in SEQ ID NO: 41.

The human IgG Fc or the variant thereof may be a fragment of an Fc including a CH3 domain or a contiguous amino acid sequence that is 90%, 92%, 95%, 96%, 97%, 98%, 99%, or 100% identical to SEQ ID NO: 41. In certain embodiments, the human IgG Fc or the variant thereof may be a fragment of an Fc including a CH2 domain and a CH3 domain or a contiguous amino acid sequence that is 90%, 92%, 95%, 96%, 97%, 98%, 99%, or 100% identical to SEQ ID NO: 41. In certain embodiments, the human IgG Fc or the variant thereof may be a fragment of an Fc including a partial hinge region, a CH2 domain, and a CH3 domain or a contiguous amino acid sequence that is 90%, 92%, 95%, 96%, 97%, 98%, 99%, or 100% identical to SEQ ID NO: 41. In certain embodiments, the human IgG Fc or the variant thereof may have an amino acid sequence that is 90%, 92%, 95%, 96%, 97%, 98%, 99%, or 100% identical to SEQ ID NO: 41.

The IgG Fc or the variant thereof includes a first polypeptide including an IgG1 Fc sequence, the IgG1 Fc sequence including a CH3 sequence including at least one engineered protuberance, and a second polypeptide including an IgG1 Fc sequence, the IgG1 Fc sequence including a CH3 sequence including at least one engineered cavity, in which the first polypeptide may be heterodimerized with the second polypeptide via positioning of the protuberance of the first polypeptide into the cavity of the second polypeptide.

Specifically, the first polypeptide may include an engineered protuberance that enables binding of another IgG Fc polypeptide (e.g. a second polypeptide) including an engineered cavity. The second polypeptide may include an engineered cavity that enables binding of another IgG Fc polypeptide (e.g. a first polypeptide) including an engineered protuberance. In addition, the protuberance of the first polypeptide and the cavity of the second polypeptide may be engineered into a CH3 domain of IgG Fc. Here, the protuberance of the first polypeptide and the cavity of the second polypeptide are neither linked nor bound to the GDF15 variant.

The engineered protuberance may include at least one substitution in the amino acid sequence of human IgG1 Fc having the amino acid sequence set forth in SEQ ID NO: 41. Here, the numbering of amino acid positions is based on an EU numbering scheme. The substitution may be present at a position selected from the group consisting of amino acid residues 347, 366, and 394. For example, the substitution may be any one selected from the group consisting of Q347W/Y, 1366W/Y, 1394W/Y, and combinations thereof. In addition, the engineered cavity may include at least one substitution of the corresponding amino acid in the human IgG1 Fc sequence, and the substitution may be present at a position selected from the group consisting of amino acid residues 366, 368, 394, 405, and 407. For example, the substitution may be any one selected from the group consisting of T366S, L368A, T394S, F405T/V/A, Y407T/V/A, and combinations thereof.

Preferably, the protuberance includes a 1366W/Y substitution, and the cavity includes any one substitution selected from the group consisting of T366S, L368A, Y407T/V/A, and combinations thereof. For example, the protuberance may include a 1366W/Y substitution and the cavity may include a Y407T/V/A substitution. Moreover, the protuberance may include a T366Y substitution and the cavity may include a Y407T substitution. The protuberance may include a T366W substitution and the cavity may include a Y407A substitution. The protuberance may include a T394Y substitution and the cavity may include a Y407T substitution.

The first polypeptide may include any one amino acid sequence selected from among SEQ ID NOs: 42, 44, and 46, and the second polypeptide may include any one amino acid sequence selected from among SEQ ID NOs: 43, 45, and 47.

The protuberance is referred to as a “knob” and the cavity is referred to as a “hole”.

The first polypeptide is an Fc ‘knob’ including an engineered protuberance, and the second polypeptide is an Fc ‘hole’ including an engineered protuberance. The first polypeptide and the second polypeptide may be physically bound to each other through either or both of non-covalent interactions (e.g. hydrophobic effects such as hydrophobic interactions between knob and hole regions of Fc) and covalent bonds (e.g. disulfide bonds, such as 1 or 2 or more disulfide bonds between hinge regions of Fc in the first polypeptide and the second polypeptide).

As used herein, the term “dimer” refers to a protein complex including at least two polypeptides. Each of these polypeptides includes an N-terminus and a C-terminus. At least two polypeptides may be linked to each other through either or both of covalent and non-covalent interactions (e.g. electrostatic effects, n-effects, van der Waals forces, and hydrophobic effects). The two polypeptides may have the same amino acid sequence or different amino acid sequences, and a complex having two polypeptides that are the same as each other is referred to as a homodimer, and a complex having two polypeptides that are different from each other is referred to as a heterodimer.

The human IgG Fc or the variant thereof may be a heterodimer including the first polypeptide and the second polypeptide, and the heterodimer may be a heterodimer using A-1 (SEQ ID NO: 42) and A-2 (SEQ ID NO: 43), a heterodimer using B-1 (SEQ ID NO: 44) and B-2 (SEQ ID NO: 45), or a heterodimer using C-1 (SEQ ID NO: 46) and C-2 (SEQ ID NO: 47).

In addition, the IgG Fc or the variant thereof may include an additional mutation in order to improve the properties of the long-acting GDF15 fusion protein. Specifically, an additional mutation may be included in the heterodimer composed of the first polypeptide and the second polypeptide.

For example, the IgG Fc or the variant thereof may include a mutation(s) that abrogates (e.g. decreases or eliminates) IgG effector function. Specifically, the Fc partner sequence may include a mutation(s) that abrogates effector functions such as complement-dependent cytotoxicity (CDC), antibody-dependent cellular cytotoxicity (ADCC), and antibody-dependent cellular phagocytosis (ADCP). For example, an IgG Fc using A-1 and A-2 or a variant (heterodimer) thereof may include E233A and L235A mutations in order to remove the IgG1 effector function. A heterodimer using B-1 and B-2 including the N297A mutation may be used to remove N-linked glycan. A heterodimer using C-1 and C-2 may include L234A, L235A, and N297A mutations in order to remove the IgG1 effector function and N-linked glycan.

The GDF15 variant and the IgG Fc or the variant thereof may be bound through binding of the C-terminus of the first polypeptide or the C-terminus of the second polypeptide of the IgG Fc or the variant thereof to the N-terminus of the GDF15 variant. In addition, the GDF15 variant and the IgG Fc or the variant thereof may be bound through binding of the N-terminus of the first polypeptide or the N-terminus of the second polypeptide of the IgG Fc or the variant thereof to the C-terminus of the GDF15 variant. Preferably, the GDF15 variant and the IgG Fc or the variant thereof are bound through binding of the C-terminus of the first polypeptide of the IgG Fc or the variant thereof to the N-terminus of the GDF15 variant.

In addition, the GDF15 variant and the IgG Fc or the variant thereof may be bound via a linker. The linker may be a peptide that includes glycine, serine, alanine, lysine and glutamic acid residues and is composed of 10 to 50 amino acid residues. The linker may include (G4S)ₙ, in which n may be an integer of 1 to 10 or an integer of 2 to 7. For example, n may be 2, 3, 4, 5, 6, or 7. In an embodiment of the present invention, a linker including (G4S)₅ in which n is an integer of 5 is used.

However, the present invention is not limited thereto, and as an example of a suitable linker other than (G4S)ₙ, the linker may include GS(G4S)ₙ, GS(EEEA)ₙ, (EEEA)ₙ, GS(EAAAK)ₙ, (EAAAK)ₙ, or GSGGSS(PT)ₙ, in which n may be an integer of 1 to 10. In an embodiment of the present invention, a linker including GS(EEEA)₆ in which n is an integer of 6 or a linker including GS(EAAAK)₅ in which n is an integer of 5 is used.

Specifically, the linker may be
(SEQ ID NO: 48)
GGGGSGGGGSGGGGSGGGGSGGGGS,
(SEQ ID NO: 92)
GSGGGGSGGGGSGGGGS,
(SEQ ID NO: 93)
GSGGGGSGGGGSGGGGSGGGGSGGGGS,
(SEQ ID NO: 94)
GSGGGGSGGGGSGGGGSGGGGSGGGGSGGGGSGGGGS,
(SEQ ID NO: 95)
GSEEEAEEEAEEEAEEEAEEEAEEEA,
(SEQ ID NO: 96)
GSGGSSPTPTPTPTPTPTPTPTPTPT,
or
(SEQ ID NO: 97)
GSEAAAKEAAAKEAAAKEAAAKEAAAK.
Preferably, the linker is
(SEQ ID NO: 48)
GGGGSGGGGSGGGGSGGGGSGGGGS,
(SEQ ID NO: 95)
GSEEEAEEEAEEEAEEEAEEEAEEEA,
or
(SEQ ID NO: 97)
GSEAAAKEAAAKEAAAKEAAAKEAAAK.

The long-acting GDF15 fusion protein includes one GDF15 variant per heterodimer composed of the first polypeptide and the second polypeptide. The GDF15 variant may include at least one N-linked glycan.

The long-acting GDF15 fusion protein may include i) a GDF15 variant including any one amino acid sequence selected from among SEQ ID NOs: 21 to 39, ii) a first polypeptide including any one amino acid sequence selected from among SEQ ID NOs: 42, 44, and 46, and iii) a second polypeptide including any one amino acid sequence selected from among SEQ ID NOs: 43, 45, and 47.

Preferably, the long-acting GDF15 fusion protein includes i) a GDF15 variant including any one amino acid sequence selected from among SEQ ID NOs: 21 to 39, ii) a linker including the amino acid sequence of SEQ ID NO: 48, iii) a first polypeptide including the amino acid sequence of SEQ ID NO: 42, and iv) a second polypeptide including the amino acid sequence of SEQ ID NO: 43.

More preferably, the long-acting GDF15 fusion protein includes i) a GDF15 variant including any one amino acid sequence selected from among SEQ ID NOs: 21 to 39, ii) a linker including any one amino acid sequence selected from among SEQ ID NOs: 92 to 97, iii) a first polypeptide including the amino acid sequence of SEQ ID NO: 46, and iv) a second polypeptide including the amino acid sequence of SEQ ID NO: 47.

Fusion Protein Dimer

A long-acting GDF15 fusion protein dimer includes two long-acting GDF15 fusion proteins. Specifically, the two long-acting GDF15 fusion proteins are dimerized through GDF15-GDF15 interaction to form what is called a “fusion protein dimer”.

Complex

The complex comprises a growth differentiation factor-15 (GDF15) variant and an IgG Fc, said complex being represented by the following formula (II):

IgG Fc−(L)ₘ−N-terminal extension domain−core domain (II)

wherein m is an integer of 0 or 1,

L is a linker selected from the group consisting of SEQ ID NOs: 48, 92, 93, 94, 95, 96, and 97,

IgG Fc comprises the amino acid sequence of SEQ ID NOs: 42, 44, or 46, and

N-terminal extension domain and core domain are as defined in the above.

In an embodiment, the N-terminal extension domain−core domain comprises any one amino acid sequence selected from the group consisting of SEQ ID NOs: 21 to 39.

Further, the complex comprises any one amino acid sequence selected from the group consisting of SEQ ID NOs: 50-91 and 98-109.

Nucleic Acid Molecule, Expression Vector, and Host Cell

The present invention pertains to an isolated nucleic acid molecule encoding the GDF15 variant or the long-acting GDF15 fusion protein.

As used herein, the term “isolated nucleic acid molecule” refers to a nucleic acid molecule of the present invention that has been separated from at least about 50% of proteins, lipids, carbohydrates, or other materials with which it is naturally found when total nucleic acid is isolated from the source cells, is operably linked to a polynucleotide to which it is not linked in nature, or does not occur in nature as part of a larger polynucleotide sequence. Specifically, the isolated nucleic acid molecule of the present invention is substantially free from any other contaminating nucleic acid molecules or other contaminants that are found in the natural environment thereof that would interfere with the use thereof for polypeptide production or for related treatment, diagnosis, prevention, or research.

As such, the isolated nucleic acid molecule encoding the GDF15 variant or the long-acting GDF15 fusion protein may have different sequences due to codon redundancy. In addition, the isolated nucleic acid molecule may be appropriately modified depending on the purpose, so long as it is able to produce the GDF15 variant or the long-acting GDF15 fusion protein, or nucleotides may be added at the N-terminus or C-terminus thereof.

The present invention pertains to an expression vector including the isolated nucleic acid molecule encoding the GDF15 variant or the long-acting GDF15 fusion protein.

As used herein, the term “expression vector” refers to a vector containing a nucleic acid sequence suitable for transformation of a host cell and directing or controlling the expression of an inserted heterologous nucleic acid sequence. Examples of the vector include, but are not limited to, linear nucleic acids, plasmids, phagemids, cosmids, RNA vectors, viral vectors, and analogues thereof. Examples of such viral vectors include, but are not limited to, retroviruses, adenoviruses, and adeno-associated viruses.

As used herein, the term “expression of a heterologous nucleic acid sequence” or “expression” of a protein of interest refers to transcription of an inserted DNA sequence, translation of an mRNA transcript, and production of a fusion protein product or antibody or antibody fragment.

A useful expression vector may be RcCMV (Invitrogen, Carlsbad) or a variant thereof. A useful expression vector may include a human cytomegalovirus (CMV) promoter to promote continuous transcription of a gene of interest in mammalian cells and a bovine growth hormone polyadenylation signal sequence to increase the steady-state level of RNA after transcription.

The present invention pertains to a host cell including the expression vector.

As used herein, the term “host cell” refers to prokaryotic and eukaryotic cells into which the recombinant expression vector may be introduced. As used herein, the terms “transformed” and “transfected” refer to the introduction of a nucleic acid (e.g. a vector) into a cell through many techniques known in the art.

The host cell may be transformed or transfected with the DNA sequence of the present invention, and may be used for expression and/or secretion of a protein of interest. The host cell that may be used in the present invention may include immortal hybridoma cells, NS/0 myeloma cells, 293 cells, Chinese hamster ovary cells (CHO cells), HeLa cells, CAP cells (cells derived from human amniotic fluid), or COS cells.

Pharmaceutical Composition

The pharmaceutical composition according to the present invention may be administered through any route. The composition of the present invention may be provided to an animal either directly (e.g. by injection, transplantation, or local administration to a tissue site, topically) or systemically (e.g. parenterally or orally) through any suitable means. When the composition of the present invention is administered via an oral or parenteral route such as intravenous, subcutaneous, ophthalmic, intraperitoneal, intramuscular, intrarectal, intraorbital, intracerebral, intracranial, intraspinal, intraventricular, intrathecal, intracisternal, intracapsular, intranasal, or aerosol administration, the pharmaceutical composition may include, for example, an aqueous or physiologically applicable suspension of body fluids or a part of the solution thereof. Accordingly, the carrier or vehicle is physiologically acceptable and thus may be added to the composition and delivered to the patient. Therefore, it is generally possible to include physiological saline as a carrier for the formulation, like a body fluid.

The frequency of administration may also vary depending on the pharmacokinetic parameters of the GDF15 variant in the formulation that is used. Typically, the clinician will administer the pharmaceutical composition until a dosage that achieves the desired effect is reached. Thus, the pharmaceutical composition may be administered in a single dose or in two or more doses at temporal intervals, or through continuous infusion using a transplantation device or catheter. Additional refinement of the appropriate dosage may be routinely made by those skilled in the art, and falls within the realm of work routinely performed thereby.

Moreover, the unit dosage in humans is 0.01 μg/kg to 100 mg/kg, particularly 1 μg/kg to 10 mg/kg body weight. Although the above amount is optimal, it may vary depending on the disease to be treated and the presence or absence of side effects, and the optimal dosage may be determined through typical experimentation. Administration of the fusion protein may be based on periodic bolus injection or continuous intravenous, subcutaneous, or intraperitoneal administration from an external reservoir (e.g. an intravenous bag) or internal reservoir (e.g. a bioerodible implant).

Administration of Composition

The composition of the present invention may be administered through any route suitable for the disease to be treated. Suitable routes include oral, parenteral (including subcutaneous, intramuscular, intravenous, intraarterial, inhalation, intradermal, intrathecal, epidural, and infusion techniques), transdermal, intrarectal, intranasal, topical (including buccal and sublingual), vaginal, intraperitoneal, intrapulmonary, and intranasal administration. Topical administration may include the use of transdermal administration, such as a transdermal patch or iontophoresis device. The formulation of drugs is disclosed in Remington's Pharmaceutical Sciences, 18^{th} Ed., (1995) Mack Publishing Co., Easton, PA. Other examples of drug formulations are described in Liberman, H. A. and Lachman, L., Eds., Pharmaceutical Dosage Forms, Marcel Decker, Vol. 3, 2^{nd} Ed., New York, NY.

The preferred routes vary depending on, for example, the condition of the recipient. For oral administration, the composition may be formulated into a pill, capsule, tablet, etc. along with a pharmaceutically acceptable carrier, lubricant, or excipient. For parenteral administration, the composition may be formulated in the form of a unit dose injection along with a pharmaceutically acceptable parenteral vehicle or diluent.

Prevention or Treatment

The composition of the present invention is used for the prevention or treatment of non-alcoholic fatty liver disease (NAFLD) or non-alcoholic steatohepatitis (NASH). The composition according to the present invention has an effect of inhibition of liver fat accumulation.

“Non-alcoholic fatty liver disease” has the same meaning as non-alcoholic fatty liver and non-alcoholic hepatic steatosis. Non-alcoholic fatty liver disease includes non-alcoholic steatohepatitis (NASH) and simple fatty liver. Non-alcoholic fatty liver disease is a liver disorder characterized by predominantly antagonistic hepatic fat deposition, similar to alcoholic liver disease, in liver tissue despite no admitted drinking history. It is defined as a concept that includes simple fatty liver with good prognosis and advanced non-alcoholic steatohepatitis.

The present invention pertains to the use of the GDF15 variant, the long-acting GDF15 fusion protein, or the long-acting GDF15 fusion protein dimer for the prevention or treatment of non-alcoholic fatty liver disease (NAFLD) or non-alcoholic steatohepatitis (NASH). The present invention pertains to a method of preventing or treating non-alcoholic fatty liver disease (NAFLD) or non-alcoholic steatohepatitis (NASH) including administering to a subject the GDF15 variant, the long-acting GDF15 fusion protein, or the long-acting GDF15 fusion protein dimer.

The subject may be a subject suffering from non-alcoholic fatty liver disease or non-alcoholic steatohepatitis. Moreover, the subject may be a mammal, preferably a human.

The term “treatment” refers to any indication of success in the treatment or amelioration of an injury, pathology, or condition, including any subjective or objective parameter such as abatement, remission, diminishing of symptoms, injury, pathology or condition more tolerable to the patient, slowing of the rate of regression or decline, creation of a final point of regression that is less debilitating, and improvement of a patient's physical or mental well-being. The treatment or amelioration of symptoms may be based on any objective or subjective parameter including physical examination, neuropsychiatric examination, and/or psychiatric evaluation.

The “effective amount” is an amount generally sufficient to reduce the severity or frequency of symptoms, eliminate the symptom or underlying cause, prevent the occurrence or underlying cause of the symptom, or ameliorate or correct any damage resulting from or associated with a disease state. In some embodiments, the effective amount is a therapeutically effective amount or a prophylactically effective amount. The “therapeutically effective amount” is an amount sufficient to correct a disease state or symptom, particularly a condition or symptom associated with the disease state, or to prevent, impede, delay, or reverse the progression of a disease state or any other undesirable symptom associated in any way with the disease. The “prophylactically effective amount” is the amount of a pharmaceutical composition that, when administered to a subject, has an intended prophylactic effect, such as preventing or delaying the onset of a disease state, or reducing the likelihood of onset (or recurrence) of a disease state or related symptoms. The therapeutically effective amount of the composition according to the present invention may be the amount of the GDF15 variant, the long-acting GDF15 fusion protein, or the long-acting GDF15 fusion protein dimer supporting reduction in an observable level of a biological or medical response, such as blood glucose, insulin, triglyceride, or cholesterol levels, weight loss, or improvements in glucose tolerance, energy expenditure, or insulin sensitivity.

Product

A kit including the GDF15 variant, the long-acting GDF15 fusion protein, or the long-acting GDF15 fusion protein dimer useful for the treatment of the diseases and disorders described above may be provided. The kit includes a single container upon co-formulation in a complex formulation form. The kit includes a container containing the GDF15 variant, the long-acting GDF15 fusion protein, or the long-acting GDF15 fusion protein dimer.

The kit may further include a label or package insert provided with or affixed to the container. The term “package insert” may refer to instructions commonly included within commercial packages for therapeutic products, and contains information regarding the indications, use, dosage, administration, contraindications and/or warnings regarding the use of the therapeutic product. Suitable containers include, for example, bottles, vials, syringes, blister packs, and the like. The container may be formed from a variety of materials, such as glass or plastic. The label or package insert indicates that the composition is used for the prevention or treatment of a particular disease, such as non-alcoholic fatty liver disease or steatohepatitis. Additionally, it may further include a second container including a pharmaceutically acceptable buffer, such as bacteriostatic water for injection (BWFI), phosphate-buffered saline, Ringer's solution, and a dextrose solution. It may further include other materials desirable from a commercial and user standpoint, such as other buffers, diluents, fillers, needles, and syringes.

The kit may further include instructions for administering the GDF15 variant, the long-acting GDF15 fusion protein, or the long-acting GDF15 fusion protein dimer simultaneously, sequentially, or separately to a patient in need thereof.

A better understanding of the present invention may be obtained through the following examples. However, these examples may be modified into various other forms, and are not to be construed as limiting the scope of the present invention.

### EXAMPLE 1

### Production of Long-Acting GDF15 Fusion Protein

### Example 1.1

### Gene Cloning

For fusion carrier and linker optimization studies on two variants (FM9+Fc_hole, FM11+Fc_hole) showing excellent activity and improved purity after purification, long-acting GDF15 fusion proteins in which fusion carriers with minimized effector functions (SEQ ID NOs: 46 and 47) and various linkers (SEQ ID NOs: 92, 93, 94, 95, 96, and 97) were designed, and are shown in Table 1 below.

TABLE 1

| Material | | | |
|---|---|---|---|
| representation | | | |
| (Sequence | GDF15 | | |
| number) | Sequence change | Fusion carrier | Linker sequence |
| FM9-1 + Fc_hole | ΔN2, S64R | IgG1 Fc_knob (SEQ ID NO: 46) | GS(G4S)₃ (SEQ |
| (SEQ ID NOs: 98 | (SEQ ID NO: 28) | IgG1 Fc_hole (SEQ ID NO: 47) | ID NO: 92) |
| and 47) | | | |
| FM9-2 + Fc_hole | ΔN2, S64R | IgG1 Fc_knob (SEQ ID NO: 46) | GS(G4S)₅ (SEQ |
| (SEQ ID NOs: 99 | (SEQ ID NO: 28) | IgG1 Fc_hole (SEQ ID NO: 47) | ID NO: 93) |
| and 47) | | | |
| FM9-3 + Fc_hole | ΔN2, S64R | IgG1 Fc_knob (SEQ ID NO: 46) | GS(G4S)₇ (SEQ |
| (SEQ ID NOs: 100 | (SEQ ID NO: 28) | IgG1 Fc_hole (SEQ ID NO: 47) | ID NO: 94) |
| and 47) | | | |
| FM9-4 + Fc_hole | ΔN2, S64R | IgG1 Fc_knob (SEQ ID NO: 46) | GS(EEEA)₆ (SEQ |
| (SEQ ID NOs: 101 | (SEQ ID NO: 28) | IgG1 Fc_hole (SEQ ID NO: 47) | ID NO: 95) |
| and 47) | | | |
| FM9-5 + Fc_hole | ΔN2, S64R | IgG1 Fc_knob (SEQ ID NO: 46) | GSGGSS(PT)₁₀ |
| (SEQ ID NOs: 102 | (SEQ ID NO: 28) | IgG1 Fc_hole (SEQ ID NO: 47) | (SEQ ID NO: 96) |
| and 47) | | | |
| FM9-6 + Fc_hole | ΔN2, S64R | IgG1 Fc_knob (SEQ ID NO: 46) | GS(EAAAK)₅ (SEQ |
| (SEQ ID NOs: 103 | (SEQ ID NO: 28) | IgG1 Fc_hole (SEQ ID NO: 47) | ID NO: 97) |
| and 47) | | | |
| FM11-1 + Fc_hole | ΔN3, G4N, D5S, | IgG1 Fc_knob (SEQ ID NO: 46) | GS(G4S)₃ (SEQ |
| (SEQ ID NOs: 104 | H6T, S64R | IgG1 Fc_hole (SEQ ID NO: 47) | ID NO: 92) |
| and 47) | (SEQ ID NO: 30) | | |
| FM11-2 + Fc_hole | ΔN3, G4N, D5S, | IgG1 Fc_knob (SEQ ID NO: 46) | GS(G4S)₅ (SEQ |
| (SEQ ID NOs: 105 | H6T, S64R | IgG1 Fc_hole (SEQ ID NO: 47) | ID NO: 93) |
| and 47) | (SEQ ID NO: 30) | | |
| FM11-3 + Fc_hole | ΔN3, G4N, D5S, | IgG1 Fc_knob (SEQ ID NO: 46) | GS(G4S)₇ (SEQ |
| (SEQ ID NOs: 106 | H6T, S64R | IgG1 Fc_hole (SEQ ID NO: 47) | ID NO: 94) |
| and 47) | (SEQ ID NO: 30) | | |
| FM11-4 + Fc_hole | ΔN3, G4N, D5S, | IgG1 Fc_knob (SEQ ID NO: 46) | GS(EEEA)₆ (SEQ |
| (SEQ ID NOs: 107 | H6T, S64R | IgG1 Fc_hole (SEQ ID NO: 47) | ID NO: 95) |
| and 47) | (SEQ ID NO: 30) | | |
| FM11-5 + Fc_hole | ΔN3, G4N, D5S, | IgG1 Fc_knob (SEQ ID NO: 46) | GSGGSS(PT)₁₀ |
| (SEQ ID NOs: 108 | H6T, S64R | IgG1 Fc_hole (SEQ ID NO: 47) | (SEQ ID NO: 96) |
| and 47) | (SEQ ID NO: 30) | | |
| FM11-6 + Fc_hole | ΔN3, G4N, D5S, | IgG1 Fc_knob (SEQ ID NO: 46) | GS(EAAAK)₅ (SEQ |
| (SEQ ID NOs: 109 | H6T, S64R | IgG1 Fc_hole (SEQ ID NO: 47) | ID NO: 97) |
| and 47) | (SEQ ID NO: 30) | | |

Specifically, in order to produce a first polypeptide (FM series) having the structure of an Fc_knob-(G4S)₅-GDF15 variant and a second polypeptide having an Fc_hole structure, gene cloning was performed using a pcDNA3.3 (Invitrogen) expression vector including a gene encoding a first polypeptide including any one amino acid sequence selected from among SEQ ID NOs: 98 to 109 and a gene encoding a second polypeptide including the amino acid sequence of SEQ ID NO: 47. Here, synthesis of the nucleotide sequences encoding the amino acid sequences of SEQ ID NOs: 98 to 109 and SEQ ID NO: 47 was outsourced to Macrogen.

### Example 1.2

### Expression and Purification of Long-Acting GDF15 Fusion Protein (Dimer)

On the 8^{th} day after transient transfection of an ExpiCHO cell line (Invitrogen) with the pcDNA3.3 expression vector cloned in Example 1.1, the cell culture fluid was harvested and purified. In order to purify the first and second polypeptides in the harvested cell culture fluid (HCCF), affinity purification was performed using Protein A resin.

Specifically, the harvested cell culture fluid was loaded on a MabSelect SuRe Protein A resin (GE Healthcare) equilibrated with 1×PBS (pH 7.4) to thus induce binding. After completion of binding of the first polypeptide and the second polypeptide, the MabSelect SuRe Protein A resin was washed with 1×PBS (pH 7.4), followed by elution using a 0.1 M glycine (pH 3.5) solution to obtain a final material.

The first polypeptide and the second polypeptide were neutralized to a pH of about 8.0 using a 1 M Tris-HCl solution. The first and second polypeptides were fully dimerized through a knock-in-hole interaction, and the result was named a “long-acting GDF15 fusion protein”. Two long-acting GDF15 fusion protein molecules were dimerized again through GDF15-GDF15 interaction, and the result was named a “fusion protein dimer”.

Also, in order to obtain a highly pure long-acting GDF15 fusion protein, a pool obtained after completion of first-step purification was subjected to second-step ion exchange (IEX) purification using an anion exchange (AEX) resin and a cation exchange (CEX) resin.

Specifically, for anion exchange (AEX), the pool obtained after the first step above was loaded on a POROS HQ 50 μm Strong Anion Exchange resin (Thermo Fisher) equilibrated with 1×PBS (pH 7.4) to thus induce binding. After completion of binding of the first polypeptide and the second polypeptide, the POROS HQ 50 μm Strong Anion Exchange resin was washed with 1×PBS (pH7.4), followed by concentration gradient elution using a 50 mM Tris-HCl (pH 8.0) solution containing 1 M sodium chloride to obtain a final material. Fractions having purity of 95% or higher were pooled using size-exclusion chromatography analysis.

In addition, for cation exchange (CEX), the pool obtained after the first step above was subjected to pH adjustment depending on the isoelectric point and then loaded on a POROS XS Strong Cation Exchange resin (Thermo Fisher) equilibrated with a 20 mM sodium phosphate (pH 6.5) solution to thus induce binding. After completion of binding of the first polypeptide and the second polypeptide, the POROS XS Strong Cation Exchange resin was washed with a 20 mM sodium phosphate (pH 6.5) solution, followed by concentration gradient elution using a 20 mM sodium phosphate (pH 6.5) solution containing 1 M sodium chloride to obtain a final material. Fractions having purity of 95% or more were pooled using size-exclusion chromatography analysis.

### EXAMPLE 2

### Measurement of Functional Activity of Long-Acting GDF15 Fusion Protein (Dimer)

Two variants (FM9+Fc_hole, FM11+Fc_hole) were compared and evaluated for GDF15 activity depending on the linker type and length. The GDF15 activity was measured using a Bright-Glo™ luciferase assay kit (Promega) and a GFRAL/RET/SRE-luc-overexpressing HEK293 (Human embryonic kidney 293) cell line.

Specifically, 1×10⁵ GFRAL/RET/SRE-luc-overexpressing HEK293 cells were dispended in DMEM containing 10% FBS in each well of a 96-well-plate, followed by culture at 37° C. and 5% CO₂ for 24 hours. After 24 hours, each medium of the 96-well plate was replaced with 50 μl of a serum-free medium, followed by culture at 37° C. and 5% CO₂ for 4 hours.

In addition, the long-acting GDF15 fusion protein produced in Example 1 was prepared by 3-fold serial dilution from a concentration of 2000 nM using a serum-free medium. Thereafter, 50 μl of the long-acting GDF15 fusion protein dilution was added to each well containing 50 μl of the replaced serum-free medium and the GFRAL/RET/SRE-luc cell line so that the actual concentration thereof was serially diluted 3-fold from 1000 nM, followed by reaction at 37° C. and 5% CO₂ for 4 hours. After 4 hours, each well was treated with 100 μl of a Bright-Glo™ solution prepared by adding a Bright-Glo™ buffer to a Bright-Glo™ substrate, followed by reaction at room temperature for 1 minute.

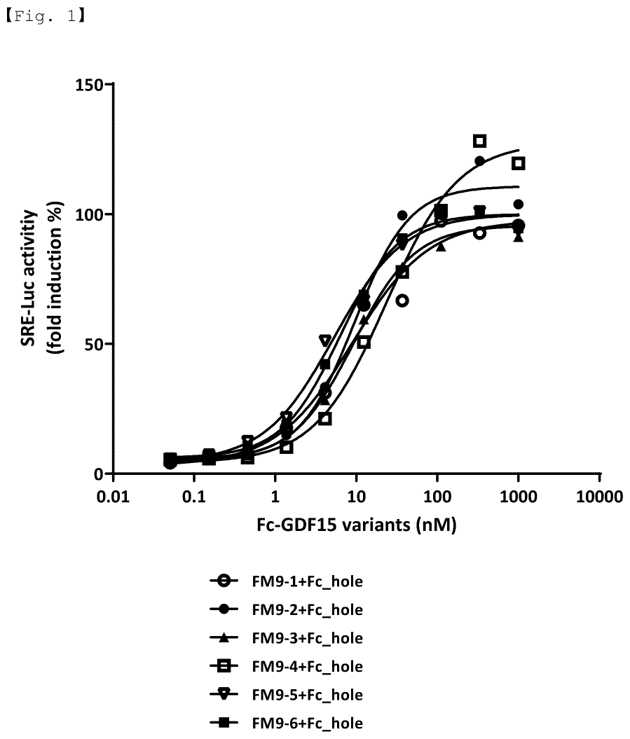

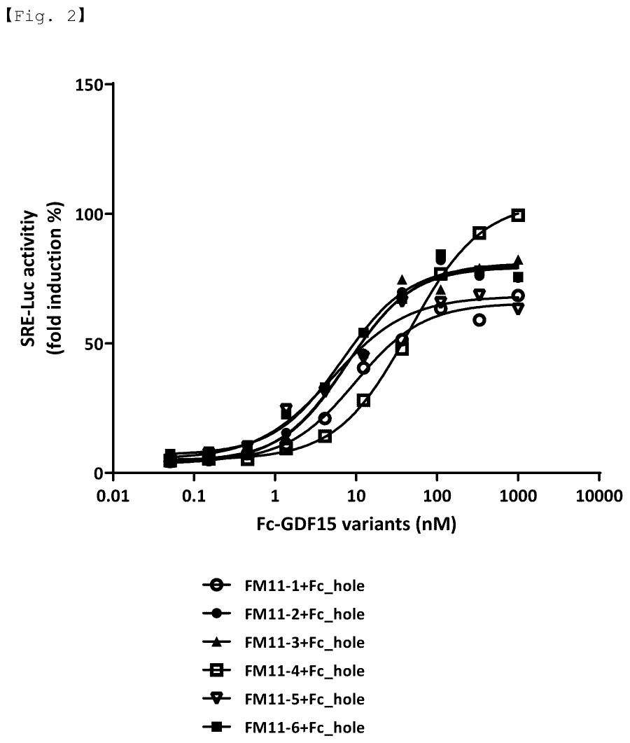

Thereafter, the reaction value (relative light units, RLU) was measured using a microplate reader (Perkin Elmer, Wallac Victor X5) capable of measuring luminescence. The results thereof are shown in Table 2 below and in FIGS. 1 and 2. Here, the functional activity of the long-acting GDF15 fusion protein depending on the GDF15 sequence, linker type, and length was compared based on the in-vitro GDF15 activity of FM9-6+Fc_hole (Eₘₐₓ 100%).

TABLE 2

| Material | GDF15 | | | | |
|---|---|---|---|---|---|
| representation | Sequence | | Linker | Eₘₐₓ | EC₅₀ |
| (Sequence number) | change | Fusion carrier | sequence | (%) | (nM) |
| FM9-1 + Fc_hole | ΔN2, S64R | IgG1 Fc_knob | GS(G4S)₃ (SEQ | 97.6 | 9.0 |
| (SEQ ID NOs: 98 and | (SEQ ID NO: | (SEQ ID NO: 46) | ID NO: 92) | | |
| 47) | 28) | IgG1 Fc_hole | | | |
| | | (SEQ ID NO: 47) | | | |
| FM9-2 + Fc_hole | ΔN2, S64R | IgG1 Fc_knob | GS(G4S)₅ (SEQ | 110.8 | 9.5 |
| (SEQ ID NOs: 99 and | (SEQ ID NO: | (SEQ ID NO: 46) | ID NO: 93) | | |
| 47) | 28) | IgG1 Fc_hole | | | |
| | | (SEQ ID NO: 47) | | | |
| FM9-3 + Fc_hole | ΔN2, S64R | IgG1 Fc_knob | GS(G4S)₇ (SEQ | 95.6 | 9.3 |
| (SEQ ID NOs: 100 | (SEQ ID NO: | (SEQ ID NO: 46) | ID NO: 94) | | |
| and 47) | 28) | IgG1 Fc_hole | | | |
| | | (SEQ ID NO: 47) | | | |
| FM9-4 + Fc_hole | ΔN2, S64R | IgG1 Fc_knob | GS(EEEA)₆ | 127.6 | 23.6 |
| (SEQ ID NOs: 101 | (SEQ ID NO: | (SEQ ID NO: 46) | (SEQ ID NO: | | |
| and 47) | 28) | IgG1 Fc_hole | 95) | | |
| | | (SEQ ID NO: 47) | | | |
| FM9-5 + Fc_hole | ΔN2, S64R | IgG1 Fc_knob | GSGGSS(PT)₁₀ | 100.0 | 5.3 |
| (SEQ ID NOs: 102 | (SEQ ID NO: | (SEQ ID NO: 46) | (SEQ ID NO: | | |
| and 47) | 28) | IgG1 Fc_hole | 96) | | |
| | | (SEQ ID NO: 47) | | | |
| FM9-6 + Fc_hole | ΔN2, S64R | IgG1 Fc_knob | GS(EAAAK)₅ | 100.0 | 6.3 |
| (SEQ ID NOs: 103 | (SEQ ID NO: | (SEQ ID NO: 46) | (SEQ ID NO: | | |
| and 47) | 28) | IgG1 Fc_hole | 97) | | |
| | | (SEQ ID NO: 47) | | | |
| FM11-1 + Fc_hole | ΔN3, G4N, | IgG1 Fc_knob | GS(G4S)₃ (SEQ | 65.6 | 9.7 |
| (SEQ ID NOs: 104 | D5S, H6T, | (SEQ ID NO: 46) | ID NO: 92) | | |
| and 47) | S64R (SEQ ID | IgG1 Fc_hole | | | |
| | NO: 30) | (SEQ ID NO: 47) | | | |
| FM11-2 + Fc_hole | ΔN3, G4N, | IgG1 Fc_knob | GS(G4S)₅ (SEQ | 79.4 | 7.7 |
| (SEQ ID NOs: 105 | D5S, H6T, | (SEQ ID NO: 46) | ID NO: 93) | | |
| and 47) | S64R (SEQ ID | IgG1 Fc_hole | | | |
| | NO: 30) | (SEQ ID NO: 47) | | | |
| FM11-3 + Fc_hole | ΔN3, G4N, | IgG1 Fc_knob | GS(G4S)₇ (SEQ | 81.0 | 7.8 |
| (SEQ ID NOs: 106 | D5S, H6T, | (SEQ ID NO: 46) | ID NO: 94) | | |
| and 47) | S64R (SEQ ID | IgG1 Fc_hole | | | |
| | NO: 30) | (SEQ ID NO: 47) | | | |
| FM11-4 + Fc_hole | ΔN3, G4N, | IgG1 Fc_knob | GS(EEEA)₆ | 104.8 | 45.4 |
| (SEQ ID NOs: 107 | D5S, H6T, | (SEQ ID NO: 46) | (SEQ ID NO: | | |
| and 47) | S64R (SEQ ID | IgG1 Fc_hole | 95) | | |
| | NO: 30) | (SEQ ID NO: 47) | | | |
| FM11-5 + Fc_hole | ΔN3, G4N, | IgG1 Fc_knob | GSGGSS(PT)₁₀ | 68.2 | 5.1 |
| (SEQ ID NOs: 108 | D5S, H6T, | (SEQ ID NO: 46) | (SEQ ID NO: | | |
| and 47) | S64R (SEQ ID | IgG1 Fc_hole | 96) | | |
| | NO: 30) | (SEQ ID NO: 47) | | | |
| FM11-6 + Fc_hole | ΔN3, G4N, | IgG1 Fc_knob | GS(EAAAK)₅ | 80.3 | 6.8 |
| (SEQ ID NOs: 109 | D5S, H6T, | (SEQ ID NO: 46) | (SEQ ID NO: | | |
| and 47) | S64R (SEQ ID | IgG1 Fc_hole | 97) | | |
| | NO: 30) | (SEQ ID NO: 47) | | | |

As is apparent from the results of Table 2, each long-acting GDF15 fusion protein showed similar activity except for the linker GS(EEEA)₆ (SEQ ID NO: 95), and the long-acting GDF15 fusion proteins (FM9-4+Fc_hole, FM11-4+Fc_hole) linked via the linker GS(EEEA)₆ (SEQ ID NO: 95) were confirmed to show relatively low EC₅₀ values and high Eₘₐₓ values (FIGS. 1 and 2).

### EXAMPLE 3

### Evaluation of Anti-Non-Alcoholic Steatohepatitis Effect of Long-Acting GDF15 Fusion Protein (Dimer) in Choline-Deficient, L-Amino-Acid-Defined, High-Fat -Diet-Induced non-Alcoholic Steatohepatitis Mouse Model (CDAHF-Diet-Induced NASH Mouse Model)

### Example 3.1

### Choline-Deficient, L-Amino-Acid-Defined, High-Fat-Diet-Induced non-Alcoholic Steatohepatitis Mouse Model (CDAHF-Diet-Induced NASH Mouse Model)

Animal models of non-alcoholic fatty liver disease (NAFLD) and non-alcoholic steatohepatitis (NASH) may be classified into four types, particularly diet-induced models, chemical-induced models, genetically modified models, and combined models. Among these, the diet-induced model containing high fat has a main pathogenesis mechanism by which excessive amounts of lipids accumulate in the liver to thus cause fatty liver, and this model is similar to human NAFLD and is the most common animal model of NAFLD. In particular, the CDAHF diet (choline-deficient, L-amino-acid -defined, high-fat diet) is a non-alcoholic fatty liver inducer that is much stronger than fructose, and accelerates the accumulation of lipids and cholesterol in the liver, resulting in additional inflammatory changes and fibrosis of the liver.

5-week-old male C57BL/6J mice (Shizuoka Laboratory Center Inc., Japan) were purchased from Central Lab. Animal Inc., Seoul, Republic of Korea. The C57BL/6J mice were subjected to adaptive acclimatization with normal food (Teklad Certified Irradiated Global 18% Protein Rodent Diet, 2918C, Harlan Co., USA) for one week, and from 6 weeks of age, the CDAHF diet (choline-deficient, L-amino-acid-defined, high-fat diet, Research Diets, Cat# A06071302, USA) was fed thereto for 6 weeks and drug treatment was initiated. On the day before drug treatment, the mice were grouped based on the body weight of individual mice (n=8 per group). Thereafter, 0.1, 1, and 3 nmol/kg of FM9-6+Fc_hole were subcutaneously administered thereto every day (QD) for a total of 8 weeks. Body weight was measured twice a week during the experimental period (56 days). Here, DPBS (Dulbecco's phosphate-buffered saline, Gibco, USA) was administered for vehicle treatment. During administration of the test material, the CDAHF diet was simultaneously fed thereto for 8 weeks and thus the mice were fed with the CDAHF diet for a total of 14 weeks.

### Example 3.2

### Improvement in Body Weight, Liver Weight, and Blood Liver Function Indicators in CDAHF-Diet-Induced NASH Mouse Model

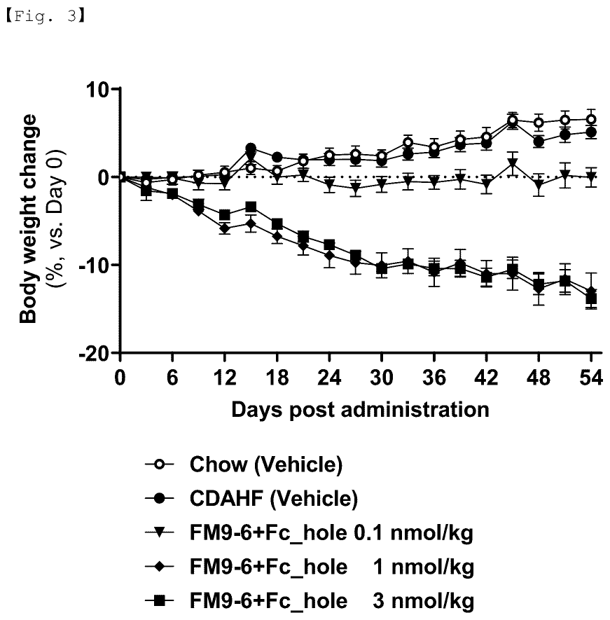

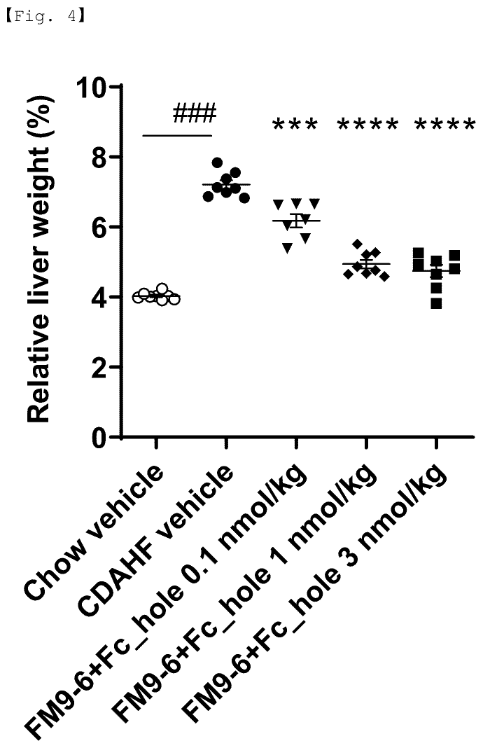

Based on the results of repeated administration of FM9-6+Fc_hole for 8 weeks, compared to the CDAHF diet control (CDAHF vehicle) (5.1%), the weight loss effect was about −0.04%*** in the group administered with 0.1 nmol/kg of FM9-6+Fc_hole, about −13.0%**** in the group administered with 1 nmol/kg of FM9-6+Fc_hole, and about −13.8%**** in the group administered with 3 nmol/kg of EM9-6+Fc_hole (***p<0.001, and ****p<0.0001 vs. CDAHF diet control (CDAHF vehicle); using a two-way ANOVA method) (FIG. 3). Moreover, when the relative liver weight (%) was evaluated in consideration of fasting body weight, it was confirmed that the relative liver weight was decreased depending on the dose of FM9-6+Fc_hole (FIG. 4).

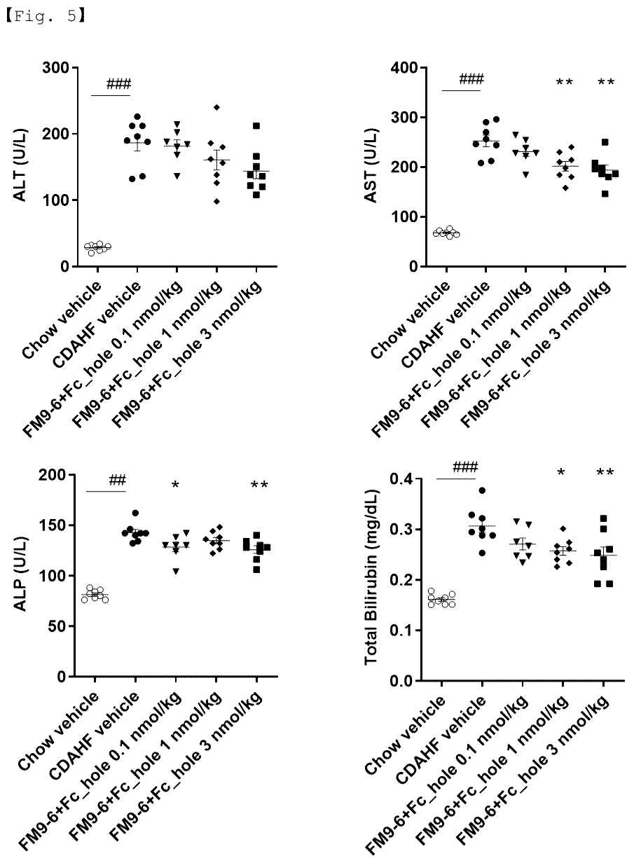

When liver function indicators in the blood were observed, levels of ALT (alanine aminotransferase), AST (aspartate aminotransferase), ALP (alkaline phosphatase), and total bilirubin were significantly increased compared to a normal control (Chow vehicle) in a CDAHF-diet-induced NASH mouse model (CDAHF vehicle). When FM9-6+Fc_hole was administered, the liver function indicators were found to decrease in a dose-dependent manner. Furthermore, statistically significant reductions in levels of AST and total bilirubin were confirmed in the group administered with 1 nmol/kg of FM9-6+Fc_hole, and statistically significant reductions in levels of ALT, AST, ALP, and total bilirubin levels were confirmed in the group administered with 3 nmol/kg of FM9-6+Fc_hole (FIG. 5).

### Example 3.3

### Histological Improvement in CDAHF-Diet-Induced NASH Mouse Model

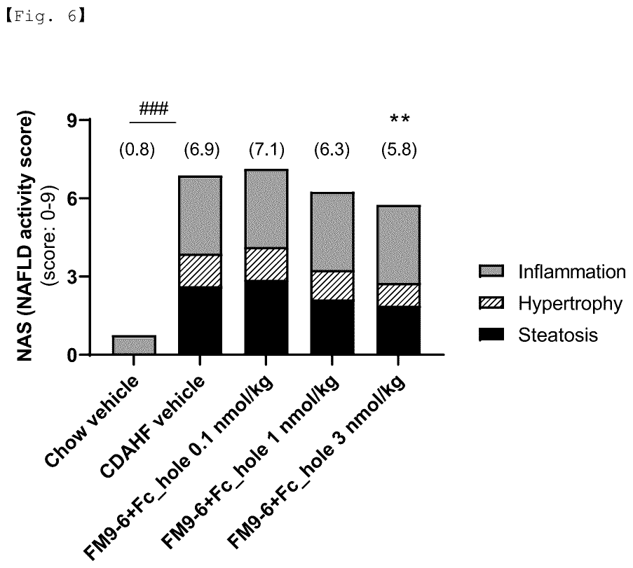

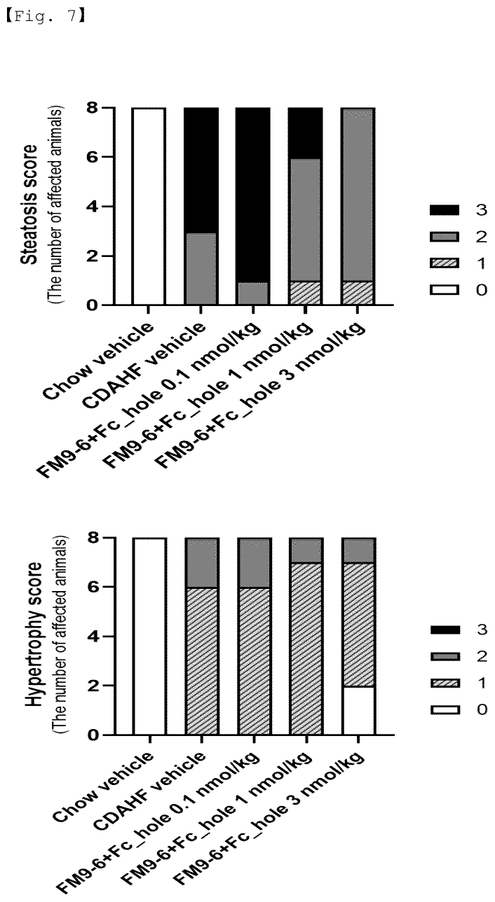

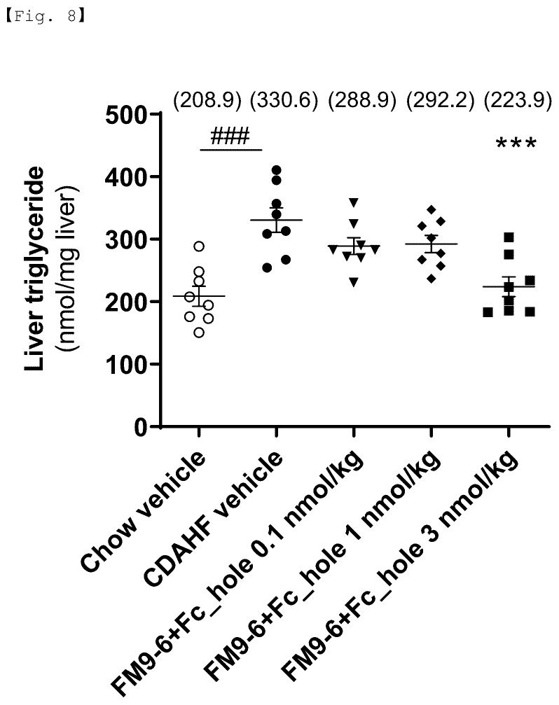

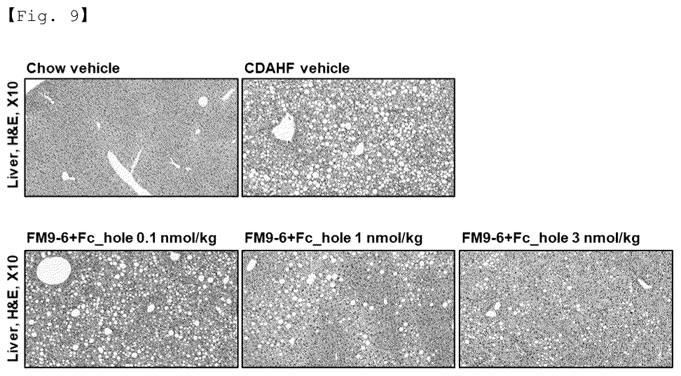

The NAFLD activity score (NAS) was significantly increased in the CDAHF-diet-induced NASH mice (CDAHF vehicle) compared to the normal control (Chow vehicle), and when 3 nmol/kg of FM9-6+Fc_hole was administered, a significant NAS reduction effect was confirmed (FIG. 6). Based on the results of individual scores of NAS, it was confirmed that the steatosis score and the hypertrophy score were lower in the groups administered with 1 and 3 nmol/kg of FM9-6+Fc_hole than in the CDAHF diet control (CDAHF vehicle) (FIG. 7). When triglyceride levels in liver tissue were analyzed, the liver triglyceride level was increased about 1.7 times in the CDAHF diet control compared to the normal control, and was decreased in a dose-dependent manner by administration of FM9-6+Fc_hole, and in particular, statistical significance appeared in the groups administered with 1 and 3 nmol/kg of FM9-6+Fc_hole (FIG. 8). In addition, as shown in the H&E-stained images of liver tissue, steatosis reduction was visually observed in the groups administered with 1 and 3 nmol/kg of FM9-6+Fc_hole (FIG. 9).

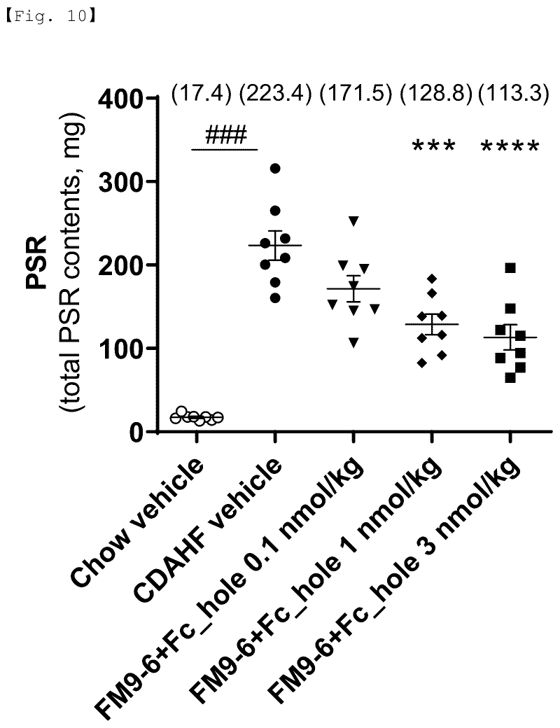

A PSR (picrosirius red) staining analysis method is a method for staining collagen in liver tissue, and total PSR content (mg) is shown in FIG. 10 in consideration of the liver weight of each individual. A significant increase in PSR was confirmed in the CDAHF diet control (CDAHF vehicle) compared to the normal control (Chow vehicle), and total PSR content was decreased in a dose-dependent manner by administration of FM9-6+Fc_hole, which means that the progression of liver fibrosis is inhibited through administration of FM9-6+Fc_hole. Statistical significance appeared in the groups administered with 1 and 3 nmol/kg of FM9-6+Fc_hole (FIG. 10).

Consequently, in the CDAHF-diet-induced NASH mouse model, administration of FM9-6+Fc_hole was found to inhibit the progression of hepatic steatosis and liver fibrosis, indicating that non-alcoholic steatohepatitis was ameliorated.

### EXAMPLE 4

### Evaluation of Anti-Non-Alcoholic Steatohepatitis Effect of Long-Acting GDF15 Fusion Protein (Dimer) in Ob/Ob Mouse Model (Model Having Obesity Caused by Genetic Modification and Hepatic Steatosis)

### Example 4.1

### Ob/Ob Mouse Model

The production and removal of fat within liver cells are regulated by various genes, and mutation, deletion, overexpression, or modification thereof may affect the metabolism of fat to thus induction fatty liver. Therefore, it is possible to create NAFLD animal models by artificially manipulating animal genes.

In particular, the ob/ob mouse model is a genetically modified model from which the leptin gene has been removed, and obesity naturally occurs because appetite suppression by leptin is not regulated, which leads to hepatic steatosis due to insulin resistance.

5-week-old male ob/ob (B6.Cg-Lep^{ob}/J) mice and C57BL/6J mice (The Jackson laboratory, USA) were purchased from Raon Bio (Yong-in, Republic of Korea). The ob/ob mice and C57BL/6J mice were fed with normal food (Teklad Certified Irradiated Global 18% Protein Rodent Diet, 2918C, Harlan Co., USA) until the end of adaptive acclimatization and study, and drug treatment was initiated after 4 weeks of adaptive acclimatization. On the day before drug treatment, the mice were grouped based on the body weight of individual mice (n=6 per group). Thereafter, 1 nmol/kg of FM9-6+Fc_hole and 30 nmol/kg of semaglutide (long-acting GLP-1 derivative) as a control were subcutaneously administered thereto every 3 days (Q3D) for a total of 4 weeks. Body weight was measured at least twice a week during the experimental period (26 days). Here, DPBS (Dulbecco's phosphate-buffered saline, Gibco, USA) was administered for vehicle treatment.

### Example 4.2

### Improvement in Body Weight, Liver Weight, and Liver Function Blood Indicators in Ob/Ob Mouse Model

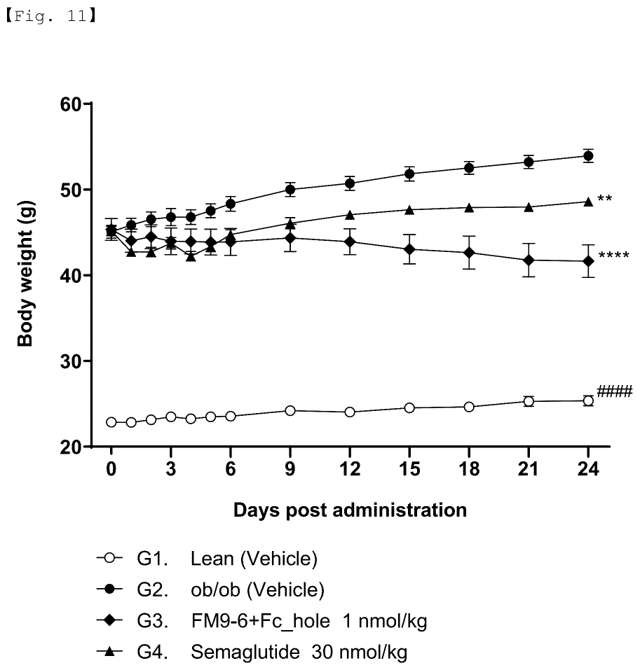

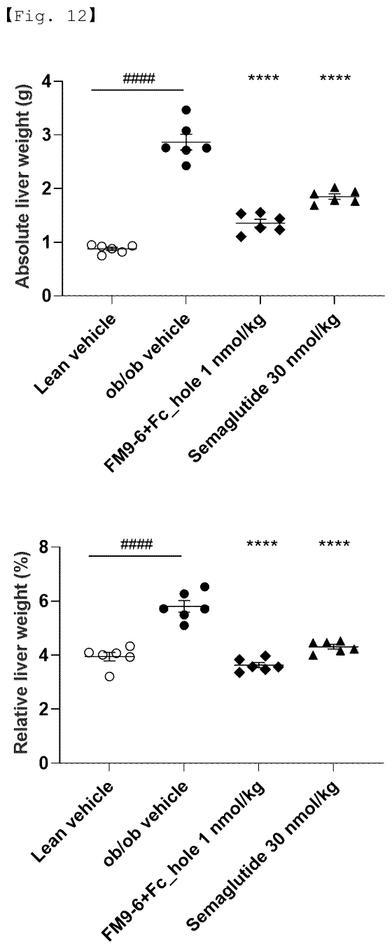

Based on the results of repeated administration of FM9-6+Fc_hole for 4 weeks, compared to the ob/ob control (ob/ob vehicle) (19.67%), the weight loss effect was about −8.24%**** in the group administered with 1 nmol/kg of FM9-6+Fc_hole, and about 7.66%** in the group administered with 30 nmol/kg of semaglutide (long-acting GLP-1 derivative) (**p<0.01, and ****p<0.0001 vs. ob/ob control (ob/ob vehicle); using a two-way ANOVA method) (FIG. 11). Moreover, when the relative liver weight (%) was evaluated in consideration of liver weight (g) and fasting body weight, a relative liver weight reduction was confirmed in the group administered with FM9-6+Fc_hole (FIG. 12).

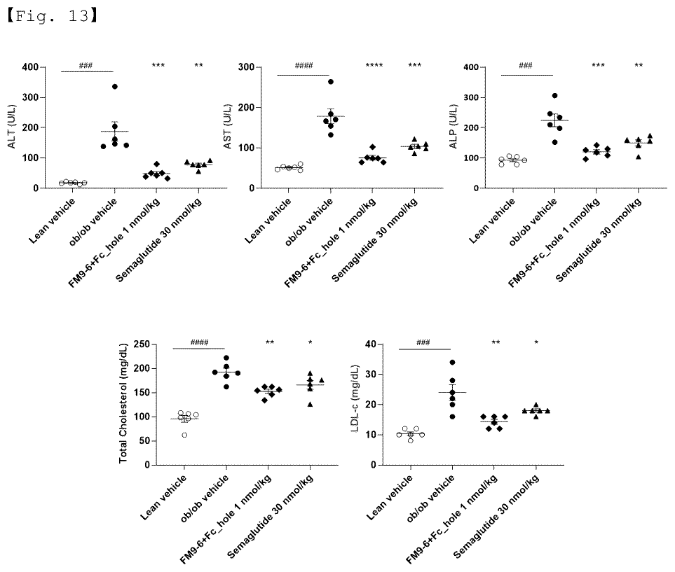

When the liver function indicators in the blood were observed, ALT, AST, ALP, total cholesterol, and LDL-c (low-density lipoprotein-cholesterol) levels were significantly increased in the ob/ob mice (ob/ob vehicle) compared to the normal control (lean vehicle). When FM9-6+Fc_hole was administered, the liver function indicators were found to decrease, and statistically significant reductions in ALT, AST, ALP, total cholesterol, and LDL-c levels were confirmed in the group administered with 1 nmol/kg of FM9-6+Fc_hole (FIG. 13).

### INDUSTRIAL APPLICABILITY

According to the present invention, a composition including a GDF15 variant, a long-acting GDF15 fusion protein, or a long-acting GDF15 fusion protein dimer is effective at preventing or treating non-alcoholic fatty liver disease (NAFLD) or non-alcoholic steatohepatitis (NASH).

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents thereof.

What is claimed is:

1. A pharmaceutical composition for preventing or treating non-alcoholic fatty liver disease (NAFLD) or non-alcoholic steatohepatitis (NASH), comprising a GDF15 (growth differentiation factor-15) variant represented by Formula (I) below, a long-acting GDF15 fusion protein, or a long-acting GDF15 fusion protein dimer as an active ingredient:
N-terminal extension domain−core domain Formula (I)
in Formula (I),
the N-terminal extension domain is a polypeptide comprising any one amino acid sequence selected from among SEQ ID NOs: 3 to 5; and
the core domain is a polypeptide comprising an amino acid sequence of SEQ ID NO: 20, or is a polypeptide in which any one selected from the group consisting of a 15^{th} amino acid, 50^{th} amino acid, 58^{th} amino acid, 97^{th} amino acid, and combinations thereof in the amino acid sequence of SEQ ID NO: 20 is substituted with another amino acid,
in which arginine (R), which is the 15^{th} amino acid, is substituted with alanine (A), aspartic acid (D), asparagine (N), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), or valine (V),
asparagine (N), which is the 50^{th} amino acid, is substituted with alanine, arginine (R), aspartic acid, cysteine, glutamic acid, glutamine, glycine, histidine, isoleucine, leucine, lysine, methionine, phenylalanine, proline, serine, threonine, tryptophan, tyrosine, or valine,
serine (S), which is the 58^{th} amino acid, is substituted with alanine, arginine, aspartic acid, asparagine, cysteine, glutamic acid, glutamine, glycine, histidine, isoleucine, leucine, lysine, methionine, phenylalanine, proline, threonine, tryptophan, tyrosine, or valine, or
aspartic acid (D), which is the 97^{th} amino acid, is substituted with alanine, arginine, asparagine, cysteine, glutamic acid, glutamine, glycine, histidine, isoleucine, leucine, lysine, methionine, phenylalanine, proline, serine, threonine, tryptophan, tyrosine, or valine.

2. The pharmaceutical composition according to claim 1, wherein the core domain comprises any one mutation selected from the group consisting of mutations (1) to (6) below:
(1) arginine (R), which is the 15^{th} amino acid in the amino acid sequence of SEQ ID NO: 20, is substituted with asparagine (N);
(2) asparagine (N), which is the 50^{th} amino acid in the amino acid sequence of SEQ ID NO: 20, is substituted with leucine (L);
(3) serine (S), which is the 58^{th} amino acid in the amino acid sequence of SEQ ID NO: 20, is substituted with lysine (K), arginine (R), asparagine (N), aspartic acid (D), glutamic acid (E), cysteine (C), or leucine (L);
(4) aspartic acid (D), which is the 97^{th} amino acid in the amino acid sequence of SEQ ID NO: 20, is substituted with leucine (L);
(5) asparagine (N), which is the 50^{th} amino acid, and aspartic acid (D), which is the 97^{th} amino acid, in the amino acid sequence of SEQ ID NO: 20, are substituted with cysteine (C) or serine (S); and
(6) arginine (R), which is the 15^{th} amino acid in the amino acid sequence of SEQ ID NO: 20, is substituted with asparagine (N), and serine (S), which is the 58^{th} amino acid, is substituted with lysine (K) or arginine (R).

3. The pharmaceutical composition according to claim 2, wherein the core domain comprises any one amino acid sequence selected from among SEQ ID NOs: 6 to 19.

4. The pharmaceutical composition according to claim 1, wherein the GDF15 variant comprises an N-terminal extension domain comprising an amino acid sequence set forth in SEQ ID NO: 3 and a core domain comprising an amino acid sequence set forth in SEQ ID NO: 8, 9, or 20.

5. The pharmaceutical composition according to claim 1, wherein the GDF15 variant comprises an N-terminal extension domain comprising an amino acid sequence set forth in SEQ ID NO: 4 and a core domain comprising an amino acid sequence set forth in SEQ ID NO: 8, 9, or 20.

6. The pharmaceutical composition according to claim 1, wherein the GDF15 variant comprises an N-terminal extension domain comprising an amino acid sequence set forth in SEQ ID NO: 5 and a core domain comprising any one amino acid sequence selected from among SEQ ID NOs: 6 to 19.

7. The pharmaceutical composition according to claim 1, wherein the GDF15 variant comprises any one amino acid sequence selected from among SEQ ID NOs: 21 to 39.

8. The pharmaceutical composition according to claim 1, wherein the long-acting GDF15 fusion protein is configured such that the GDF15 variant and a human IgG Fc or a variant thereof are bound to each other.

9. The pharmaceutical composition according to claim 1, wherein the long-acting GDF15 fusion protein dimer comprises two long-acting GDF15 fusion proteins.

10. The pharmaceutical composition according to claim 8, wherein the IgG Fc or the variant thereof comprises:
a first polypeptide comprising an IgG1 Fc sequence, the IgG1 Fc sequence comprising a CH3 sequence comprising at least one engineered protuberance; and
a second polypeptide comprising an IgG1 Fc sequence, the IgG1 Fc sequence comprising a CH3 sequence comprising at least one engineered cavity,
wherein the first polypeptide and the second polypeptide are heterodimerized via positioning of the protuberance of the first polypeptide into the cavity of the second polypeptide.

11. The pharmaceutical composition according to claim 8, wherein a C-terminus of a first polypeptide or a C-terminus of a second polypeptide of the IgG Fc or the variant thereof is bound to an N-terminus of the GDF15 variant.

12. The pharmaceutical composition according to claim 8, wherein the GDF15 variant and the IgG Fc or the variant thereof are bound via a linker.

13. The pharmaceutical composition according to claim 12, wherein the linker is a peptide comprising glycine, serine, alanine, lysine, and glutamic acid residues and composed of 10 to 50 amino acid residues.

14. The pharmaceutical composition according to claim 12, wherein the linker is GGGGSGGGGSGGGGSGGGGSGGGGS (SEQ ID NO: 48), GSGGGGSGGGGSGGGGS (SEQ ID NO: 92), GSGGGGSGGGGSGGGGSGGGGSGGGGS (SEQ ID NO: 93), GSGGGGSGGGGSGGGGSGGGGSGGGGSGGGGSGGGGS (SEQ ID NO: 94), GSEEEAEEEAEEEAEEEAEEEAEEEA (SEQ ID NO: 95), GSGGSSPTPTPTPTPTPTPTPTPTPT (SEQ ID NO: 96), or GSEAAAKEAAAKEAAAKEAAAKEAAAK (SEQ ID NO: 97).

15. The pharmaceutical composition according to claim 10, wherein the first polypeptide comprises any one amino acid sequence selected from among SEQ ID NOs: 42, 44, and 46.

16. The pharmaceutical composition according to claim 10, wherein the second polypeptide comprises any one amino acid sequence selected from among SEQ ID NOs: 43, 45, and 47.

17. The pharmaceutical composition according to claim 1, wherein the GDF15 variant comprises at least one N-linked glycan (N-linked glycan).

18. The pharmaceutical composition according to claim 1, wherein the long-acting GDF15 fusion protein comprises i) a GDF15 variant comprising any one amino acid sequence selected from among SEQ ID NOs: 21 to 39, ii) a first polypeptide comprising any one amino acid sequence selected from among SEQ ID NOs: 42, 44, and 46, and iii) a second polypeptide comprising any one amino acid sequence selected from among SEQ ID NOs: 43, 45, and 47.

19. A pharmaceutical composition for preventing or treating non-alcoholic fatty liver disease (NAFLD) or non-alcoholic steatohepatitis (NASH), comprising a complex comprising a growth differentiation factor-15 (GDF15) variant and an IgG Fc, said complex being represented by the following formula (II):
IgG Fc−(L)ₘ−N-terminal extension domain−core domain (II)
wherein m is an integer of 0 or 1,
L is a linker selected from the group consisting of SEQ ID NOs: 48, 92, 93, 94, 95, 96, and 97,
IgG Fc comprises the amino acid sequence of SEQ ID NOs: 42, 44, or 46, and
N-terminal extension domain and core domain are as defined in claim 1.

20. The pharmaceutical composition according to claim 19, wherein the N-terminal extension domain−core domain comprises any one amino acid sequence selected from the group consisting of SEQ ID NOs: 21 to 39.

21. The pharmaceutical composition according to claim 19, wherein the complex comprises any one amino acid sequence selected from the group consisting of SEQ ID NOs: 50-91 and 98-109.
